SEQUENCE LISTING (5 sequences)

SEQ ID NO: 1 - protein, 580 aa, Homo sapiens
  misc_feature: Mucolipin-1
MTAPAGPRGSETERLLTPNPGYGTQAGPSPAPPTPPEEEDLRRRLKYFFMSPCDKFRAKGRKPCKLMLQVVKILVVTVQLILFGLSNQLAVTFREENTIAFRHLFLLGYSDGADDTFAAYTREQLYQAIFHAVDQYLALPDVSLGRYAYVRGGGDPWTNGSGLALCQRYYHRGHVDPANDTFDIDPMVVTDCIQVDPPERPPPPPSDDLTLLESSSSYKNLTLKFHKLVNVTIHFRLKTINLQSLINNEIPDCYTFSVLITFDNKAHSGRIPISLETQAHIQECKHPSVFQHGDNSFRLLFDVVVILTCSLSFLLCARSLLRGFLLQNEFVGFMWRQRGRVISLWERLEFVNGWYILLVTSDVLTISGTIMKIGIEAKNLASYDVCSILLGTSTLLVWVGVIRYLTFFHNYNILIATLRVALPSVMRFCCCVAVIYLGYCFCGWIVLGPYHVKFRSLSMVSECLFSLINGDDMFVTFAAMQAQQGRSSLVWLFSQLYLYSFISLFIYMVLSLFIALITGAYDTIKHPGGAGAEESELQAYIAQCQDSPTSGKFRRGSGSACSLLCCCGRDPSEEHSLLVN

SEQ ID NO: 2 - DNA, 2084 bp, Homo sapiens
  misc_feature: mucolipin 1 (MCOLN1)
acagatcagctgatgccggagggtttgaagccgcgccgcgagggagcgaggtcgcagtgacagcggcgggcgatcggacccaggctgccccgccgtacccgcctgcgtcccgcgctcccgccccagcatgacagccccggcgggtccgcgcggctcagagaccgagcggcttctgacccccaaccccgggtatgggacccaggcggggccttcaccggcccctccgacacccccagaagaggaagaccttcgccgtcgtctcaaatactttttcatgagtccctgcgacaagtttcgagccaagggccgcaagccctgcaagctgatgctgcaagtggtcaagatcctggtggtcacggtgcagctcatcctgtttgggctcagtaatcagctggctgtgacattccgggaagagaacaccatcgccttccgacacctcttcctgctgggctactcggacggagcggatgacaccttcgcagcctacacgcgggagcagctgtaccaggccatcttccatgctgtggaccagtacctggcgttgcctgacgtgtcactgggccggtatgcgtatgtccgtggtgggggtgacccttggaccaatggctcagggcttgctctctgccagcggtactaccaccgaggccacgtggacccggccaacgacacatttgacattgatccgatggtggttactgactgcatccaggtggatccccccgagcggccccctccgccccccagcgacgatctcaccctcttggaaagcagctccagttacaagaacctcacgctcaaattccacaagctggtcaatgtcaccatccacttccggctgaagaccattaacctccagagcctcatcaataatgagatcccggactgctataccttcagcgtcctgatcacgtttgacaacaaagcacacagtgggcggatccccatcagcctggagacccaggcccacatccaggagtgtaagcaccccagtgtcttccagcacggagacaacagcttccggctcctgtttgacgtggtggtcatcctcacctgctccctgtccttcctcctctgcgcccgctcactccttcgaggcttcctgctgcagaacgagtttgtggggttcatgtggcggcagcggggacgggtcatcagcctgtgggagcggctggaatttgtcaatggctggtacatcctgctcgtcaccagcgatgtgctcaccatctcgggcaccatcatgaagatcggcatcgaggccaagaacttggcgagctacgacgtctgcagcatcctcctgggcacctcgacgctgctggtgtgggtgggcgtgatccgctacctgaccttcttccacaactacaatatcctcatcgccacactgcgggtggccctgcccagcgtcatgcgcttctgctgctgcgtggctgtcatctacctgggctactgcttctgtggctggatcgtgctggggccctatcatgtgaagttccgctcactctccatggtgtctgagtgcctgttctcgctcatcaatggggacgacatgtttgtgacgttcgccgccatgcaggcgcagcagggccgcagcagcctggtgtggctcttctcccagctctacctttactccttcatcagcctcttcatctacatggtgctcagcctcttcatcgcgctcatcaccggcgcctacgacaccatcaagcatcccggcggcgcaggcgcagaggagagcgagctgcaggcctacatcgcacagtgccaggacagccccacctccggcaagttccgccgcgggagcggctcggcctgcagccttctctgctgctgcggaagggacccctcggaggagcattcgctgctggtgaattgattcgacctgactgccgttggaccgtaggccctggactgcagagacccccgcccccgaccccgcttatttatttgtagggtttgcttttaaggatcggctccctgtcgcgcccgaggagggcctggacctttcgtgtcggacccttgggggcggggagactgggtggggagggtgttgaataaaagggaaaataaatgtgtcgttttcattttta

SEQ ID NO: 3 - protein, 8 aa, Artificial Sequence
  2A consensus sequence
  misc_feature (2)..(2): Xaa can be any naturally occurring amino acid
  misc_feature (4)..(4): Xaa can be any naturally occurring amino acid
DXEXNPGP

SEQ ID NO: 4 - DNA, 5158 bp, Artificial Sequence
  >pAAV SC.JeT.Mcoln.pA  (5158 bp)
ctgcgcgctcgctcgctcactgaggccgcccgggcaaagcccgggcgtcgggcgacctttggtcgcccggcctcagtgagcgagcgagcgcgcagagagggagtggttaagctagcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtgtccaggcggccgccaccatgacagccccggcgggtccgcgcggctcagagaccgagcggcttctgacccccaaccccgggtatgggacccaggcggggccttcaccggcccctccgacacccccagaagaggaagaccttcgccgtcgtctcaaatactttttcatgagtccctgcgacaagtttcgagccaagggccgcaagccctgcaagctgatgctgcaagtggtcaagatcctggtggtcacggtgcagctcatcctgtttgggctcagtaatcagctggctgtgacattccgggaagagaacaccatcgccttccgacacctcttcctgctgggctactcggacggagcggatgacaccttcgcagcctacacgcgggagcagctgtaccaggccatcttccatgctgtggaccagtacctggcgttgcctgacgtgtcactgggccggtatgcgtatgtccgtggtgggggtgacccttggaccaatggctcagggcttgctctctgccagcggtactaccaccgaggccacgtggacccggccaacgacacatttgacattgatccgatggtggttactgactgcatccaggtggatccccccgagcggccccctccgccccccagcgacgatctcaccctcttggaaagcagctccagttacaagaacctcacgctcaaattccacaagctggtcaatgtcaccatccacttccggctgaagaccattaacctccagagcctcatcaataatgagatcccggactgctataccttcagcgtcctgatcacgtttgacaacaaagcacacagtgggcggatccccatcagcctggagacccaggcccacatccaggagtgtaagcaccccagtgtcttccagcacggagacaacagcttccggctcctgtttgacgtggtggtcatcctcacctgctccctgtccttcctcctctgcgcccgctcactccttcgaggcttcctgctgcagaacgagtttgtggggttcatgtggcggcagcggggacgggtcatcagcctgtgggagcggctggaatttgtcaatggctggtacatcctgctcgtcaccagcgatgtgctcaccatctcgggcaccatcatgaagatcggcatcgaggccaagaacttggcgagctacgacgtctgcagcatcctcctgggcacctcgacgctgctggtgtgggtgggcgtgatccgctacctgaccttcttccacaactacaatatcctcatcgccacactgcgggtggccctgcccagcgtcatgcgcttctgctgctgcgtggctgtcatctacctgggctactgcttctgtggctggatcgtgctggggccctatcatgtgaagttccgctcactctccatggtgtctgagtgcctgttctcgctcatcaatggggacgacatgtttgtgacgttcgccgccatgcaggcgcagcagggccgcagcagcctggtgtggctcttctcccagctctacctttactccttcatcagcctcttcatctacatggtgctcagcctcttcatcgcgctcatcaccggcgcctacgacaccatcaagcatcccggcggcgcaggcgcagaggagagcgagctgcaggcctacatcgcacagtgccaggacagccccacctccggcaagttccgccgcgggagcggctcggcctgcagccttctctgctgctgcggaagggacccctcggaggagcattcgctgctggtgaattaataagcttaataaaagatctttattttcattagatctgtgtgttggttttttgtgtgctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 5 - DNA, 5411 bp, Artificial Sequence
  >pAAV SC.hSyn1.Mcoln.pA  (5411 bp)
ctgcgcgctcgctcgctcactgaggccgcccgggcaaagcccgggcgtcgggcgacctttggtcgcccggcctcagtgagcgagcgagcgcgcagagagggagtggttaagctagcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggtgtccaggcggccgccaccatgacagccccggcgggtccgcgcggctcagagaccgagcggcttctgacccccaaccccgggtatgggacccaggcggggccttcaccggcccctccgacacccccagaagaggaagaccttcgccgtcgtctcaaatactttttcatgagtccctgcgacaagtttcgagccaagggccgcaagccctgcaagctgatgctgcaagtggtcaagatcctggtggtcacggtgcagctcatcctgtttgggctcagtaatcagctggctgtgacattccgggaagagaacaccatcgccttccgacacctcttcctgctgggctactcggacggagcggatgacaccttcgcagcctacacgcgggagcagctgtaccaggccatcttccatgctgtggaccagtacctggcgttgcctgacgtgtcactgggccggtatgcgtatgtccgtggtgggggtgacccttggaccaatggctcagggcttgctctctgccagcggtactaccaccgaggccacgtggacccggccaacgacacatttgacattgatccgatggtggttactgactgcatccaggtggatccccccgagcggccccctccgccccccagcgacgatctcaccctcttggaaagcagctccagttacaagaacctcacgctcaaattccacaagctggtcaatgtcaccatccacttccggctgaagaccattaacctccagagcctcatcaataatgagatcccggactgctataccttcagcgtcctgatcacgtttgacaacaaagcacacagtgggcggatccccatcagcctggagacccaggcccacatccaggagtgtaagcaccccagtgtcttccagcacggagacaacagcttccggctcctgtttgacgtggtggtcatcctcacctgctccctgtccttcctcctctgcgcccgctcactccttcgaggcttcctgctgcagaacgagtttgtggggttcatgtggcggcagcggggacgggtcatcagcctgtgggagcggctggaatttgtcaatggctggtacatcctgctcgtcaccagcgatgtgctcaccatctcgggcaccatcatgaagatcggcatcgaggccaagaacttggcgagctacgacgtctgcagcatcctcctgggcacctcgacgctgctggtgtgggtgggcgtgatccgctacctgaccttcttccacaactacaatatcctcatcgccacactgcgggtggccctgcccagcgtcatgcgcttctgctgctgcgtggctgtcatctacctgggctactgcttctgtggctggatcgtgctggggccctatcatgtgaagttccgctcactctccatggtgtctgagtgcctgttctcgctcatcaatggggacgacatgtttgtgacgttcgccgccatgcaggcgcagcagggccgcagcagcctggtgtggctcttctcccagctctacctttactccttcatcagcctcttcatctacatggtgctcagcctcttcatcgcgctcatcaccggcgcctacgacaccatcaagcatcccggcggcgcaggcgcagaggagagcgagctgcaggcctacatcgcacagtgccaggacagccccacctccggcaagttccgccgcgggagcggctcggcctgcagccttctctgctgctgcggaagggacccctcggaggagcattcgctgctggtgaattaataagcttaataaaagatctttattttcattagatctgtgtgttggttttttgtgtgctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

(19) United States
(12) Patent Application Publication
(10) Pub. No.: US 2022/0372520 A1
(43) Pub. Date: Nov. 24, 2022

(54) Gene Therapy Approaches to Mucolipidosis IV (MLIV)

(71) Applicant: The General Hospital Corporation, Boston, MA (US)

(72) Inventors: Yulia Grishchuk, Charlestown, MA (US); Susan A. Slaugenhaupt, Hingham, MA (US)

(21) Appl. No.: 17/772,017

(22) PCT Filed: Oct. 29, 2020
(86) PCT No.: PCT/US2020/057839

§ 371 Date: Apr. 26, 2022

Related U.S. Application Data
(60) Provisional application No. 62/927,538, filed on Oct. 29, 2019.

(51) Int. Cl. C12N 15/861; A61K 48/00

(52) U.S. Cl. CPC C12N 15/861; A61K 48/005; A61K 48/0075; C12N 2830/008

(57) ABSTRACT

Described herein are compositions comprising AAV vectors comprising a sequence encoding mucolipin 1, and methods of use thereof for gene therapy of Mucolipidosis IV (MLIV).

### CLAIM OF PRIORITY

This application claims the benefit of U.S. Provisional Application Ser. No. 62/927,538, filed on Oct. 29, 2019. The entire contents of the foregoing are incorporated herein by reference.

### TECHNICAL FIELD

Described herein are compositions comprising AAV vectors comprising a sequence encoding mucolipin 1, and methods of use thereof for gene therapy of Mucolipidosis IV (MLIV).

### BACKGROUND

Mucolipidosis type IV (MLIV) is a developmental disorder that causes motor and cognitive deficiencies, which become noticeable during the first year of life. On average, maximal neurological development in MLIV-affected individuals corresponds to the level of 15-18 months of age and remains stable throughout the third decade of life (21). In most affected individuals, neurological symptoms include spasticity, hypotonia, an inability to walk independently, ptosis, myopathic facies, drooling, difficulties in chewing and swallowing, and severely impaired fine-motor function (21). Ophthalmic symptoms are progressive and result in blindness in the second decade of life (22; 23; 24). Although mucolipidosis IV was previously considered limited to Ashkenazi Jews, the disease is pan-ethnic and patients from different other ethnic groups are known. At present there is no therapy for MLIV.

### SUMMARY

Described herein are adeno-associated (scAAV) viral vectors comprising a sequence encoding Mucolipin-1 (MCOLN1, also known as TRPML1) protein, operably linked to a promoter that drives expression of the MCOLN1 protein in a cell. Preferably, the AAV is a self-complementary scAAV or single-stranded AAV-PHP.b.

In some embodiments, the AAV further comprises one or more cis-regulatory elements that increase expression of the MCOLN1, e.g., one or more enhancers; posttranscriptional regulatory elements; cell penetrating peptides; polyadenylation sequences; and/or an intron. In some embodiments, the posttranscriptional regulatory element is HBV Posttranscriptional Regulatory Element (HPRE) or woodchuck hepatitis virus post-transcriptional regulatory element (WPRE), or a variant thereof In some embodiments, the intron is SV40 intron, F.IX truncated intron 1; β-globin SD/immunoglobin heavy chain SA; Adenovirus SD/immunoglobulin SA; SV40 late SD/SA (19S/16S); Hybrid adenovirus SD/IgG SA; or minute virus of mice (MVM) intron.

In some embodiments, the MCOLN1 protein is at least 80%, 85%, 90%, 95%, 97%, or 100% identical to SEQ ID NO:1.

In some embodiments, the promotor is a ubiquitous promoter, preferably Jet promoter, or a neuron-specific promoter, preferably a synapsin I (Syn1) promoter.

In some embodiments, the AAV is AAV9 serotype (or comprises a capsid from AAV9) and comprises or consists of (or consists essentially of, i.e., does not include other sequences that affect expression of the MCOLN1 transgene) a JeT promoter or a Syn1 promoter and polyadenylation sequence.

In some embodiments, the AAV also includes an antibiotic resistance gene, e.g., kanamycin.

Also provided are compositions and host cells comprising the AAV.

Also provided herein are methods for treating mucolipidosis IV (MLIV) in a subject, by administering to the subject a therapeutically effective amount of an AAV as described herein, as well as the AAVs for use in treating MLIV in a subject. In some embodiments, the AAV is administered or formulated to be administrated to the subject by direct administration into the CNS or eye of the subject. In some embodiments, administration into the CNS is by intrathecal or intracerebroventicular injection.

In some embodiments, the subject has developed one or more symptoms of mucolipidosis IV selected from spasticity, hypotonia, an inability to walk independently, ptosis, myopathic facies, drooling, difficulties in chewing and swallowing, impaired fine-motor function, and progressive blindness. In some embodiments, the one or more symptoms of mucolipidosis IV are improved.

In some embodiments, the AAV is administered once, twice, three times, or more, optionally wherein the AAV is administered once weekly, once monthly, biweekly, bimonthly, every three months, every four months, every five months, every six months, or annually.

Provided herein are compositions comprised of an AAV expression vector constructed with a hMCOLN1 expression cassette insert, preferably including a JeT or SYN1 promoter, as described herein. In some embodiments, the AAV vector is self-complementary AAV9, single-stranded AAV-PHP.b, AAV9, or Anc80.

Also provided herein are methods to prevent, treat or slow the progress of lysosomal storage disorders in a patient, by gene transfer of MCOLN1 to said patient by means of any of the vector compositions described herein, wherein wild type (non-mutated) MCOLN1 is expressed in the biological organs/tissues of said patient. In some embodiments, the biological organs/tissues are brain and liver. In some embodiments, the lysosomal storage disease is mucolipidosis IV.

Alost provided herein are methods to prevent, treat or slow the progress of lysosomal storage disorders in a patient comprised of a combined interventional approach wherein the first intervention is the gene transfer of MCOLN1 to said patient by means of a vector compositions described herein, wherein wild type (non-mutated) MCOLN1 is expressed in the biological organs/tissues of said patient

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Pre-symptomatic administration of AAV-PHP.b-MCOLN1 prevents onset of early motor deficits in Mcoln1^{-/-} mice. A, B. Vertical activity measurements in the open field test, represented as vertical counts and vertical time in either central (A) or total area (B) of the open field arena, show significant impairment in the saline-treated 2 months-old Mcoln1^{-/-} mice that was fully prevented in the Mcoln1^{-/-} mice treated with AAV-PHP.b-MCOLN1 intravenously at the age of 5-6 weeks; n (WT SALINE)=9; n (HET SALINE)=18; n (KO SALINE)=9; n (KO PHP.b-MCOLN1)=14. Data presented as mean values and SEM; group comparisons made using unpaired T-test. C. qRT-PCR analysis of the MCOLN1 transcripts showing transgene overexpression in the cerebral cortex, cerebellum, liver and quad muscle. Data presented as mean values and SEM; n (WT SALINE)=9; n (HET SALINE)=6-7; n (KO SALINE)=5; n (KO PHP.b-MCOLN1)=7. D. qRT-PCR analysis of the myelination marker Mbp, microgliosis marker Cd68 and astrocytosis marker Gfap in the cerebral cortex; n (HET SALINE)=7; n (KO SALINE)=5; n (KO PHP.b-MCOLN1)=7. Data presented as mean values and SEM, group comparisons were made using un-paired T-test.

FIGS. 2A-E. Systemic administration of AAV-PHP.b-MCOLN1 in symptomatic Mcoln1^{-/-} mice restores motor function and significantly extends time to paralysis. A, B. Measurements of the vertical activity in the open field test show significantly impaired activity, represented as vertical counts and vertical time in either central (A) or total area (B) of the open field arena, in the 4 months-old Mcoln1^{-/-} mice treated with saline, and its significant recovery in the Mcoln1^{-/-} mice that were treated with AAV-PHP.b-MCOLN1 at the symptomatic stage of the disease at 2 months of age; n (HET SALINE)=15; n (KO SALINE)=12; n (KO PHP.b-MCOLN1)=9. Data presented as mean values and SEM; group comparisons made using un-paired T-test. C, D. Significant long-term improvement of the rotarod performance presented as a probability to retain on the rod (C) or average latency to fall (D) in the Mcoln1^{-/-} mice treated with AAV-PHP.b-MCOLN1. C. Log-rank analysis of the probability-to-retain-on-the-rod curves revealed significant changes between curves at the age of 4 (p=0.036) and 5 months (p=0.0052) due to worse rotarod performance of the KO SALINE group, and no changes between HET SALINE and KO PHP.b-MCOLN1 curves at 6, 7, 8, 9, 10 months; at 6 mo KO SALINE mice developed hind-limbs weakness and had to be excluded from testing; at 7 mo all the KO SALINE mice were euthanized due to paralysis; n (HET SALINE)=14; n (KO SALINE)=12; n (KO PHP.b-MCOLN1)=9. D. Two-way ANOVA test (treatment group×age) of the average latency to fall for KO SALINE and KO PHP.b-MCOLN1 groups at 4 and 5 mo time points shows significant effect of AAV treatment, p=0.0007; F=13.62. E. Systemic administration of AAV PHP.b-MCOLN1 significantly delays time to paralysis in Mcoln1^{-/-} mice. The criterion for paralysis was loss of righting reflex when mouse failed to rotate itself in upright position after placing on a side within 10 sec; n (HET SALINE)=14; n (KO SALINE)=12; n (KO PHP.b-MCOLN1)=9; log-rank test p-value is <0.0001.

FIGS. 3A-D. Biodistribution and efficacy of systemic administration of AAV-PHP.b-MCOLN1 in symptomatic Mcoln1^{-/-} mice. A. qRT-PCR analysis of the MCOLN1 transcripts showing transgene overexpression in the cerebral cortex, cerebellum, liver, quad muscle and sciatic nerve. Data presented as mean values and SEM; n (HET SALINE)=14; n (KO SALINE)=12; n (KO PHP.b-MCOLN1)=9. B. qRT-PCR analysis of the myelination marker Mbp shows partial recovery in the PHP.b-MCOLN1-treated Mcoln1^{-/-} mice. Data presented as mean values and SEM; n (HET SALINE)=14; n (KO SALINE)=12; n (KO PHP.b-MCOLN1)=9; group comparisons were made using unpaired T-test. C. qRT-PCR analysis of the microgliosis marker Cd68 and astrocytosis marker Gfap in the cerebral cortex shows increased glial activation in the sham-treated Mcoln1^{-/-} mice that was not changed in the PHP.b-MCOLN1-treated Mcoln1^{-/-} mice; n (HET SALINE)=14; n (KO SALINE)=12; n (KO PHP.b-MCOLN1)=9. Data presented as mean values and SEM, group comparisons were made using unpaired T-test. D. No significant weight changes were observed in either experimental group. Data presented as median values and interquartile range, n (HET SALINE)=14; n (KO SALINE)=12; n (KO PHP.b-MCOLN1)=9.

FIGS. 4A-E. The biodistribution and efficacy of intracerebroventricular delivery of scAAV9-JeT-MCOLN1 in Mcoln1^{-/-} neonatal mice. A. qRT-PCR analysis of the MCOLN1 transcripts demonstrates dose-dependent expression of MCOLN1 in cerebral cortex, cerebellum, liver, quad muscle, and sciatic nerve. Data presented as mean values and SEM; n (HET SALINE)=3-7; n (KO SALINE)=2-5; n (KO scAAV9-JeT-MCOLN1; 2e10 vg/mouse)=5-7; n (KO scAAV9-JeT-MCOLN1; 1e10 vg/mouse)=16; n (KO scAAV9-JeT-MCOLN1; 4e9 vg/mouse)=12. B, C. Vertical activity measured in the open field test as vertical counts and vertical time in either central (B) or total area (C) of the open field arena is significantly improved in Mcoln1^{-/-} mice treated with the highest dose of scAAV9-JeT-MCOLN1 (2e10 vg/mouse) injected ICV on post-natal day 1. Data presented as mean values and SEM; n (HET SALINE)=20; n (KO SALINE)=10; n (KO scAAV9-JeT-MCOLN1; 2e10 vg/mouse)=7; n (KO scAAV9-JeT-MCOLN1; 1e10 vg/mouse)=5; n (KO scAAV9-JeT-MCOLN1; 4e9 vg/mouse)=10; group comparisons made using unpaired T-test. D. ICV delivery of scAAV9-JeT-MCOLN1 reduces LAMP-positive lysosomal aggregates in the brain of Mcoln1^{-/-} mice as demonstrated by LAMP immunohistochemistry representative images and blinded quantitative analysis on the right. LAMP staining presented as average particle size and percent of LAMP-positive area. Data presented as mean values and SEM, group comparisons made using unpaired T-test; n (HET SALINE)=4; n (KO SALINE)=5; n (KO scAAV9-JeT-MCOLN1; 2e10 vg/mouse)=4. E. ICV delivery of scAAV9-JeT-MCOLN1 improves corpus callosum thickness in the Mcoln1^{-/-} mice. Representative images of MBP staining in the left panel show thicker corpus callosum (white selection). Blinded quantitative image analysis (right panel) shows enlarged area of the MBP-positive corpus callosum. Data presented as mean values and SEM, group comparisons made using unpaired T-test; n (HET SALINE)=6; n (KO SALINE)=5; n (KO scAAV9-JeT-MCOLN1; 2e10 vg/mouse)=5.

FIGS. 5A-C. Neuron-specific expression of MCOLN1 is sufficient to rescue early motor dysfunction in Mcoln1^{-/-} mice. A, B. Vertical activity measured in the open field test as vertical counts and vertical time in either central (A) or total area (B) of the open field arena is significantly improved in Mcoln1^{-/-} mice treated with the scAAV9-SYN1-MCOLN1 (neuron-specific expression) similarly to scAAV9-JeT-MCOLN1 (ubiquitous expression). Both vectors were injected ICV at 2×10 vg/mouse on post-natal day 1. Data presented as mean values and SEM; n (HET SALINE)=20; n (KO SALINE)=10; n (KO scAAV9-JeT-MCOLN1; 2×10 vg/mouse)=7; n (KO scAAV9-SYN1-MCOLN1; 2×10 vg/mouse)=10; group comparisons made using unpaired T-test. C. qRT-PCR analysis of the MCOLN1 transcripts shows expression of the transgene in cerebral cortex, cerebellum, liver, quad muscle, and sciatic nerve. Data presented as mean values and SEM; n (HET SALINE)=4-5; n (KO SALINE)=4-5; n (KO scAAV9-JeT-MCOLN1; 2e10 vg/mouse)=6; n (KO scAAV9-SYN1-MCOLN1; 2e10 vg/mouse)=4-6. Group comparisons made using unpaired T-test.

FIGS. 6A-F. Brain targeting is required to achieve the therapeutic effect of MCOLN1 gene transfer. A. qRT-PCR analysis of the MCOLN1 transcripts shows that intravenous administration of scAAV9-JeT-MCOLN1 leads to high expression of MCOLN1 in peripheral tissues and very low expression in the brain. Data presented as mean values and SEM; n (HET SALINE)=7; n (KO SALINE)=10; n (KO scAAV9-JeT-MCOLN1)=9. B, C. Vertical activity deficits are not rescued in Mcoln1^{-/-} mice after intravenous administration scAAV9-JeT-MCOLN1. Vertical activity is presented as vertical counts and vertical time in either central (B) or total area (C) of the open field arena. N (HET SALINE)=16; n (KO SALINE)=10; n (KO scAAV9-JeT-MCOLN1)=9. Data presented as mean values and SEM; group comparisons made using un-paired T-test. D, E. Rotarod test data presented either as a probability to retain on the rod (D) or average latency to fall (E) revealed no improvement in performance of the Mcoln1^{-/-} mice treated with scAAV9-JeT-MCOLN1 intravenously. F. No significant weight changes have been observed in either experimental group. Data presented as median values and interquartile range, n (HET SALINE)=16; n (KO SALINE)=10; n (KO scAAV9-JeT-MCOLN1)=9.

FIG. 7. Open field test reveals robust motor deficits in Mcoln1^{-/-} mice. Data of the first 15 min of exploration in the arena are shown. Male and female Mcoln1^{-/-} mice demonstrate significantly reduced vertical activity presented in the form of vertical counts and vertical time in the center of the arena and in the total area of the arena. N, males: (WT, 1 mo)=13, (KO, 1 mo)=9, (WT, 2 mo)=17, (KO, 2 mo)=17, (WT, 4 mo)=14, (KO, 4 mo)=9, (WT, 5 mo)=7, (KO, 5 mo)=8; females: (WT, 1 mo)=8, (KO, 1 mo)=8, (WT, 2 mo)=22, (KO, 2 mo)=36, (WT, 4 mo)=28, (KO, 4 mo)=28, (WT, 5 mo)=10, (KO, 5 mo)=8.

FIGS. 8A-B. Exemplary design of scAAV vector with constitutive (JeT, 8A) or neuron-specific (hSyn1, 8B) promoter.

### DETAILED DESCRIPTION

Mucolipidosis IV (MLIV) is a neurodevelopmental disorder caused by aberrant lysosomal storage of lipids and water-soluble substances, generally caused by a transport defect in the late steps of endocytosis, resulting in a defect in organization of white matter in the brain and reduced maintenance of cells in the retina and optic nerve. See, e.g., Schiffmann et al., Mucolipidosis IV 2005 Jan. 28 [Updated 2015 Jul. 30]. In: Adam M P, Ardinger H H, Pagon R A, et al., editors. GeneReviews® [Internet]. Seattle (Wash.): University of Washington, Seattle; 1993-2020. Available from: ncbi.nlm.nih.gov/books/NBK1214/.

Described herein are compositions comprising viral vectors for use in treatment of subjects with MLIV, to deliver sequences encoding MCOLN1 to rescue, reduce, or reverse deficits caused by the disease.

Methods of Treatment

The methods described herein include methods for the treatment of Mucolipidosis IV (MLIV). Generally, the methods include administering a therapeutically effective amount of a vector comprising a sequence encoding MCOLN1 as described herein, to a subject who is in need of, or who has been determined to be in need of, such treatment.

MLIV causes neurodegeneration in some individuals, with reduced life expectancy as compared to healthy individuals. Typically, subjects present with severe psychomotor delay by the end of the first year of life, as well as (sometimes later-developing) visual impairment caused by a combination of corneal clouding and retinal degeneration. A diagnosis can be made based on typical clinical findings and elevated plasma gastrin concentration (normal range is 0-200 pg/mL) or polymorphic lysosomal inclusions in skin or conjunctival biopsy, or based on detection of the presence of biallelic pathogenic variants in the MCOLN1 gene. Methods for detecting pathogenic variants include exome sequencing, genome sequencing, and mitochondrial sequencing, sequence analysis, deletion/duplication analysis, gene-targeted deletion/duplication analysis or a genotyping assay specifically designed to detect deletions (e.g., breakpoint PCR or allele-specific primer extension) can be employed.

The following table presents a non-limiting list of known pathogenic mutations in MCOLN1.

| DNA change (cDNA) | Effect on Protein |
|---|---|
| c.237 + 1G > A | p.? |
| c.406 − 2A > G | p.? |
| c.681 − 104T > G | p.(=) |
| c.694A > C | p.Thr232Pro) |
| c.707G > A | p.(Arg236Gln) |
| c.920del | p.(Leu307ProfsTer65) |
| c.1084G > T | p.(Asp362Tyr) |
| c.1221_1223delCTT | p.Phe408del) |
| c.1406A > G | p. (Phe454_Asn569del) |
| 6.4-kb deletion beginning in the 5′UTR and extending into exon 6 | |

The present methods and compositions can be used in subjects who have symptoms, as well as pre-symptomatic individuals, e.g., identified by genetic screening before symptoms develop.

As used in this context, to “treat” means to ameliorate at least one symptom of MLIV. Often, MLIV results in neurological symptoms including spasticity, hypotonia, an inability to walk independently, ptosis, myopathic facies, drooling, difficulties in chewing and swallowing, and severely impaired fine-motor function, and ophthalmic symptoms including progressive blindness in the second decade of life; thus, a treatment comprising administration of a therapeutically effective amount of a compound described herein can result in a reduction in one or more of these symptoms, or a delay or reduction in risk of development of one or more of these symptoms.

Preferably, the vectors are administered by direct injection into the CNS, e.g., by intracranial (e.g., intrathecal or intracerebroventicular) administration, and/or by ocular administration (e.g., intraocular injection or ocular topical application). The methods can be administered, once, twice, three times, or more, e.g., on a routine schedule, e.g., once weekly, once monthly, biweekly, bimonthly, every three months, every four months, every five months, every six months, or annually. The methods include administering a therapeutically effective dose; in some embodiments, the dose in the range of 5×10\hat{ }13 vg−5×10\hat{ }14 vg.

Mucolipin-1 (MCOLN1)

The methods and compositions described herein provide for delivery of sequences encoding MCOLN1. For treatment of human subjects, a sequence encoding human MCOLN1 is preferably used. An exemplary sequence of human MCOLN1 protein is provided in GenBank at RefSeq ID NP_065394.1, reproduced here as SEQ ID NO:1.

Mucolipin-1, Homo sapiens
(SEQ ID NO: 1)

| | |
|---|---|
| 1 | MTAPAGPRGS ETERLLTPNP GYGTQAGPSP APPTPPEEED |
| | LRRRLKYFFM SPCDKFRAKG |
| 61 | RKPCKLMLQV VKILVVTVQL ILFGLSNQLA VTFREENTIA |
| | FRHLFLLGYS DGADDTFAAY |
| 121 | TREQLYQAIF HAVDQYLALP DVSLGRYAYV RGGGDPWTNG |
| | SGLALCQRYY HRGHVDPAND |
| 181 | TFDIDPMVVTDCIQVDPPER PPPPPSDDLTLLESSSSYKN |
| | LTLKFHKLVN VTIHFRLKTI |
| 241 | NLQSLINNEI PDCYTFSVLI TFDNKAHSGR IPISLETQAH |
| | IQECKHPSVF QHGDNSFRLL |
| 301 | FDVVVILTCS LSFLLCARSL LRGFLLQNEF VGFMWRQRGR |
| | VISLWERLEF VNGWYILLVT |
| 361 | SDVLTISGTI MKIGIEAKNL ASYDVCSILL GTSTLLVWVG |
| | VIRYLTFFHN YNILIATLRV |
| 421 | ALPSVMRFCCCVAVIYLGYCFCGWIVLGPY HVKFRSLSMV |
| | SECLFSLINGDDMFVTFAAM |
| 481 | QAQQGRSSLV WLFSQLYLYS FISLFIYMVL SLFIALITGA |
| | YDTIKHPGGA GAEESELQAY |
| 541 | IAQCQDSPTS GKFRRGSGSA CSLLCCCGRD PSEEHSLLVN |

An exemplary nucleic acid sequence encoding human MCOLN1 is provided in GenBank at RefSeq ID NM 020533.3, reproduced here as SEQ ID NO:2.

Homo sapiens mucolipin 1 (MCOLN1)
(SEQ ID NO: 2)

| | |
|---|---|
| 1 | acagatcagc tgatgccgga gggtttgaag ccgcgccgcg |
| | agggagcgag gtcgcagtga |
| 61 | cagcggcggg cgatcggacc caggctgccc cgccgtaccc |
| | gcctgcgtcc cgcgctcccg |
| 121 | ccccagcatg acagccccgg cgggtccgcg cggctcagag |
| | accgagcggc ttctgacccc |
| 181 | caaccccggg tatgggaccc aggcggggcc ttcaccggcc |
| | cctccgacac ccccagaaga |
| 241 | ggaagacctt cgccgtcgtc tcaaatactt tttcatgagt |
| | ccctgcgaca agtttcgagc |
| 301 | caagggccgc aagccctgca agctgatgct gcaagtggtc |
| | aagatcctgg tggtcacggt |
| 361 | gcagctcatc ctgtttgggc tcagtaatca gctggctgtg |
| | acattccggg aagagaacac |
| 421 | catcgccttc cgacacctct tcctgctggg ctactcggac |
| | ggagcggatg acaccttcgc |
| 481 | agcctacacg cgggagcagc tgtaccaggc catcttccat |
| | gctgtggacc agtacctggc |
| 541 | gttgcctgac gtgtcactgg gccggtatgc gtatgtccgt |
| | ggtgggggtg acccttggac |
| 601 | caatggctca gggcttgctc tctgccagcg gtactaccac |
| | cgaggccacg tggacccggc |
| 661 | caacgacaca tttgacattg atccgatggt ggttactgac |
| | tgcatccagg tggatccccc |
| 721 | cgagcggccc cctccgcccc ccagcgacga tctcaccctc |
| | ttggaaagca gctccagtta |
| 781 | caagaacctc acgctcaaat tccacaagct ggtcaatgtc |
| | accatccact tccggctgaa |
| 841 | gaccattaac ctccagagcc tcatcaataa tgagatcccg |
| | gactgctata ccttcagcgt |
| 901 | cctgatcacg tttgacaaca aagcacacag tgggcggatc |
| | cccatcagcc tggagaccca |
| 961 | ggcccacatc caggagtgta agcaccccag tgtcttccag |
| | cacggagaca acagcttccg |
| 1021 | gctcctgttt gacgtggtgg tcatcctcac ctgctccctg |
| | tccttcctcc tctgcgcccg |
| 1081 | ctcactcctt cgaggcttcc tgctgcagaa cgagtttgtg |
| | gggttcatgt ggcggcagcg |
| 1141 | gggacgggtc atcagcctgt gggagcggct ggaatttgtc |
| | aatggctggt acatcctgct |
| 1201 | cgtcaccagc gatgtgctca ccatctcggg caccatcatg |
| | aagatcggca tcgaggccaa |
| 1261 | gaacttggcg agctacgacg tctgcagcat cctcctgggc |
| | acctcgacgc tgctggtgtg |
| 1321 | ggtgggcgtg atccgctacc tgaccttctt ccacaactac |
| | aatatcctca tcgccacact |
| 1381 | gcgggtggcc ctgcccagcg tcatgcgctt ctgctgctgc |
| | gtggctgtca tctacctggg |
| 1441 | ctactgcttc tgtggctgga tcgtgctggg gccctatcat |
| | gtgaagttcc gctcactctc |
| 1501 | catggtgtct gagtgcctgt tctcgctcat caatggggac |
| | gacatgtttg tgacgttcgc |
| 1561 | cgccatgcag gcgcagcagg gccgcagcag cctggtgtgg |
| | ctcttctccc agctctacct |
| 1621 | ttactccttc atcagcctct tcatctacat ggtgctcagc |
| | ctcttcatcg cgctcatcac |
| 1681 | cggcgcctac gacaccatca agcatcccgg cggcgcaggc |
| | gcagaggaga gcgagctgca |
| 1741 | ggcctacatc gcacagtgcc aggacagccc cacctccggc |
| | aagttccgcc gcgggagcgg |
| 1801 | ctcggcctgc agccttctct gctgctgcgg aagggacccc |
| | tcggaggagc attcgctgct |
| 1861 | ggtgaattga ttcgacctga ctgccgttgg accgtaggcc |
| | ctggactgca gagacccccg |
| 1921 | cccccgaccc cgcttattta tttgtagggt ttgcttttaa |
| | ggatcggctc cctgtcgcgc |
| 1981 | ccgaggaggg cctggacctt tcgtgtcgga cccttggggg |
| | cggggagact gggtggggag |
| 2041 | ggtgttgaat aaaagggaaa ataaatgtgt cgttttcatt |
| | ttta |

Preferably, a sequence that encodes a protein that is at least 80%, 85%, 90%, 95%, 97%, 98%, or 99% identical to the exemplary sequence (SEQ ID NO:1) is used, and that retains the activity of the exemplary sequence. Of course, no mutations identified as pathogenic (e.g., in the table above) should be included. In some embodiments a sequence that encodes a protein comprising SEQ ID NO:1, that is at least 80%, 85%, 90%, 95%, 97%, 98%, or 99% identical to the SEQ ID NO:2, is used. For example, in some embodiments, the nucleic acid sequences used in the present methods and compositions are codon-optimized for increased expression in humans. See, e.g., Fuglsang, Protein Expr Purif. 2003; 31:247-249.

### MCOLN1 Activity

Relevant activity of the exemplary (wild type) sequence includes localization of MCOLN1 or TFEB to intracellular vesicular membranes including lysosomes, functioning as a cation channel that is permeable to Ca²⁺, Fe²⁺, Na⁺, K⁺, and H⁺; and/or functioning in regulating TFEB activation, or other assays, including the following: Localization with lysosome:

MCOLN1 localization with lysosomes can be confirmed by immunocyto- or histochemical analysis of colocalization of the GFP-tagged mucolipin-1 with either the lysosomal marker LAMP1 or LysoTracker (25).

### TFEB Activation:

Functional causality between activation of MCOLN1 and TFEB is well-established and has been experimentally demonstrated in multiple cellular systems (26-30). Activation of TFEB can be measured via TFEB nuclear localization assay a-TFEB antibodies (26). TFEB nuclear translocation analysis will be performed as previously described (26, 31).

### MCOLN1 Cationic Transfer:

Fe permeability measurements (32): HEK293T cells were either transfected with EGFP-MCOLN1 alone or co-transfected with mCherry-MCOLN1 and EGFP-LAMP1 (a marker for LEL). The size of the LEL is usually, 0.5 mm, which is suboptimal for patch clamping. We therefore treated cells with 1 mM vacuolin-1 (for 1-2 h), a small chemical known to increase the size of endosomes and lysosomes selectively. Large vacuoles (up to 3 mm) were observed in most vacuolin-treated cells. Occasionally, enlarged LEL could also been obtained from TRPML1-transfected cells without vacuolin-1 treatment. No significant difference in TRPML channel properties was seen for enlarged LEL with or without vacuolin-1 treatment. The vacuoles that were positive for both mCherry-TRPML1 and EGFP-LAMP1 were considered as enlarged LEL. Whole-lysosome, lysosome-attached and lysosome luminal-side-out recordings were performed on isolated enlarged LEL, similar to what was performed in enlarged endosomes. In brief, a patch pipette (electrode) was pressed against a cell and then quickly pulled away from the cell to slice the cell membrane. Enlarged LEL were released into the dish and identified by monitoring the TRPML1-EGFP, the TRPML1-mCherry or the LAMP1-EGFP fluorescence.

Ca²⁺ Permeability (25) Measurement:

Patch-clamp: Whole-lysosome planar patch-clamp recordings and preparation of lysosomes (HEK293, Human Fibroblast) were performed as follows: late endosomes and lysosomes were enlarged by treating the cells with 1 mM vacuolin-1 overnight. The following day, cells were homogenized in homogenization buffer (0.25 M sucrose, 10 mM Tris-Cl, pH 7.4) on ice using a potter homogenizer to obtain whole-cell lysates. The lysates were centrifuged at 14,000 g for 15 min at 4 C. The supernatant was treated with 8 mM CaCl2 (final concentration) on ice for 5 min, followed by a second centrifugation step at 25,000 g for 15 min at 4 C. The supernatant was collected and the pellet washed in 150 mM KCl, 10 mM Tris-Cl, pH 7.4, followed by a final centrifugation step at 25,000 g for 15 min at 4 C. The planar patch-clamp technology combined with a pressure control system and microstructured glass chips containing an aperture of B1 mm diameter (resistances of 10-15 MO) (Port-a-Patch, Nanion Technologies) were applied. Currents were recorded using an EPC-10 patch-clamp amplifier and PatchMaster acquisition software (HEKA). Data were digitized at 40 kHz and filtered at 2.8 kHz. Cytoplasmic solution contained (in mM) 60 KF, 70 K-MSA (methanesulfonate), 0.2 Ca-MSA, 10 HEPES (pH adjusted with KOH to 7.2). Luminal solution was 60 Ca-MSA, 70 K-MSA, 10 HEPES (pH adjusted with MSA to 4.6). The membrane potential was held at [symbol] 60 mV, and 500 ms voltage ramps from 200 to [symbol] 100 mV were applied every 5 s. Due to high luminal calcium used to ensure giga seals when performing whole-endolysosomal patch-clamp on the port-a-patch system 37,38, the inward currents are less rectifying and reverse at more positive potentials, depending on current amplitude and protein expression level. All recordings were obtained at room temperature and were analysed using PatchMaster (HEKA) and Origin 6.1 (OriginLab) software. Liquid junction potential was corrected. Water-soluble diC8-PIP2, PI(3,5)P2 were purchased from A.G. Scientific. For the application of all activators, cytoplasmic (external) solution was completely exchanged by a solution containing the respective activator. The EC50 values of graded dose-response curves were fitted with the Hill equation.

Ca-imaging: Calcium imaging experiments can be performed using fura-2. HEK293 cells are plated onto glass coverslips, grown overnight and transiently transfected with the respected cDNAs using TurboFect transfection reagent (Thermo Scientific). After 24-48 h, cells are loaded for 1 h with the fluorescent indicator fura-2-AM (4 mM; Invitrogen) in a standard bath solution containing (in mM) 138 NaCl, 6 KCl, 2 MgCl2, 2 CaCl2, 10 HEPES and 5.5 D-glucose (adjusted to pH 7.4 with NaOH). Cells were washed in standard bath solution for 30 min before measurement. Calcium imaging was performed using a monochromator-based imaging system (Polychrome IV monochromator, TILL Photonics).

Percent Identity

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Vectors

Sequences encoding MCOLN1 can be delivered to a subject, e.g., to the brain of a subject, using a viral vector. Described herein are expression vectors for in vivo transfection and expression of a polynucleotide that encodes MCOLN1. Expression constructs can be administered in any effective carrier, e.g., any formulation or composition capable of effectively delivering the component gene to the cells.

An exemplary approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Viral vectors can be used as a recombinant gene delivery system for the transfer of exogenous genes in vivo, particularly into humans. These vectors provide efficient delivery of genes into cells, and in some cases the transferred nucleic acids are stably integrated into the chromosomal DNA of the host. Protocols for producing recombinant viruses and for infecting cells in vitro or in vivo with such viruses can be found in Ausubel, et al., eds., Gene Therapy Protocols Volume 1: Production and In Vivo Applications of Gene Transfer Vectors, Humana Press, (2008), pp. 1-32 and other standard laboratory manuals.

A preferred viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992); see also Domenger and Grimm, Human Molecular Genetics, 28(R1):R3-R14 (October 2019)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). AAV vectors, particularly AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Space for exogenous DNA is limited to about 4.5 kb.

A variety of nucleic acids have been introduced into different cell types using AAV vectors (see for example the references cited above and those cited in Asokan et al., Molecular Therapy (2012); 20 4, 699-708; and Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993); Hammond et al., PLoS One. 2017 Dec. 15; 12(12):e0188830; Haggert et al., Mol Ther Methods Clin Dev. 2019 Nov. 26; 17:69-82. In some embodiments, viral vectors used herein are, or comprise capsid proteins from, adeno-associated virus type 1, 2, 5, 7, 8, 9, rh.10, AAV2/1, AAVDJ8, PHP.B, or Anc80. See, e.g., US PGPub 20190100560; Hammond et al., PLoS One. 2017 Dec. 15; 12(12):e0188830; Haggert et al., Mol Ther Methods Clin Dev. 2019 Nov. 26; 17:69-82; Jackson et al., Frontiers in Molecular Neuroscience (9) (2016); doi.org/10.3389/fnmol.2016.00116; Choi et al., Curr. Gene Ther. 2005; 5: 299-310.

In some embodiments, a self-complementary AAV is used, which contains an inverted repeat genome that folds to make double-stranded DNA, and wherein the right (3′) Inverted Terminal Repeat (ITR) contains a deletion of the D-sequence (the packaging signal). See, e.g., Hirata and Russell and Russell, J. Virol. (2000) 74:4612-4620; Raj et al., Expert Rev Hematol. 2011 October; 4(5): 539-549; US 20170362608; McCarty et al., Gene Therapy. 8 (16): 1248-54 (2001); McCarty et al., Mol. Ther. 2008; 16: 1648-1656; McCarty et al., Gene Ther. 2003; 10: 2112-2118; US20020006664, US20030153519, US 20030139363; US20040029106; U.S. Pat. Nos. 9,783,824; 6,547,099; 6,506,559; and 4,766,072; PCT Application Nos. WO 2001/92551, WO 2001/68836, and WO 2003/010180.

Other viruses, e.g., retroviruses or adenovirus-derived vectors, can also be used.

The vectors preferably include inverted terminal repeats (ITRs); promoters, enhancers (e.g., CMV enhancer), other cis-regulatory elements, and/or capsid serotype variants that control and drive expression of the MCOLN1 protein. With regard to promoters, vectors can include promoters that drive expression in many cell types (e.g., human β-actin, human elongation factor-1α, chicken β-actin combined with cytomegalovirus early enhancer, cytomegalovirus (CMV), simian virus 40 (SC40), herpes simplex virus thymidine kinase (HSVTK), JeT (US20020098547), PGK, CAG, sCAG, or CASI) or specifically in neurons (e.g., synapsin I (Syn1), calcium/calmodulin-dependent protein kinase II, tubulin alpha I, neuron-specific enolase and platelet-derived growth factor beta chain promoters, and hybrid promoters created by fusing cytomegalovirus enhancer (E) to those neuron-specific promoters (Hioki et al., Gene Therapy 14:872-882 (2007)). Other cis-regulatory elements can include posttranscriptional regulatory elements; 2A enhancers; polyadenylation sequences; and/or an intron. Posttranscriptional regulatory elements can include HBV Posttranscriptional Regulatory Element (HPRE), woodchuck hepatitis virus post-transcriptional regulatory element (WPRE) or variants thereof (e.g., WPRE2, WPRE3 see, e.g., Kalev-Zylinska M L, During MJJ Neurosci. 2007 Sep. 26; 27(39):10456-67; Zanta-Boussif et al., Gene Therapy (16): 605-619 (2009); Choi et al., Mol Brain. 7:17 (2014); U.S. Pat. No. 6,136,597). One or more 2A sequences can be used, e.g., 18-22 aa-long peptides that share a core sequence motif of DxExNPGP (SEQ ID NO:3) and induce ribosomal skipping during translation of a protein in a cell. Exemplary polyadenylation sequences, which include SV40, human growth hormone (hGH), bovine growth hormone (bGH), synthetic polyadenylation (spA), and rbGlob, preferably include the sequence motif AAUAAA that promotes both polyadenylation and termination (Buck and Wijnholds, Int J Mol Sci. 2020 June; 21(12): 4197). Commonly used 2A sequences include P2A, E2A, F2A and T2A. F2A is derived from foot-and-mouth disease virus 18; E2A is derived from equine rhinitis A virus; P2A is derived from porcine teschovirus-1 2A; T2A is derived from thosea asigna virus 2A. See, e.g., Lewis et al., J Neurosci Methods. 2015 Dec. 30; 256: 22-29; Liu et al., Sci Rep. 2017 May 19; 7(1):2193. In some embodiments, the AAV also includes a furin cleavage sequence (see Fang et al., Nat Biotechnol 23: 584-590). Introns can include SV40 intron, F.IX truncated intron 1; β-globin SD/immunoglobin heavy chain SA; Adenovirus SD/immunoglobulin SA; SV40 late SD/SA (19S/16S); Hybrid adenovirus SD/IgG SA; or minute virus of mice (MVM) intron (see Powell and Rivera-Soto, Discov Med. 2015 January; 19(102): 49-57. Capsid variants can include PHP.B and those described in Castle et al., Methods Mol. Biol. 2016; 1382: 133-149; Davidsson et al., PNAS. 116 (52) 27053-27062 (2019); Lee et al., Current Opinion in Biomedical Engineering (7):58-63 (2018). In some embodiments, the AAV includes a cell penetrating peptide (CPP); see Liu et al., Mol Ther Methods Clin Dev. 2014; 1: 12; Tan et al., Nature Communications 10:3733 (2019). For additional disclosure regarding viral vectors, see also Li and Samulski, Nature Reviews Genetics 21:255-272 (2020); Nair et al., iScience. 2020 Mar. 27; 23(3):100888; Haery et al., Front Neuroanat. 2019; 13: 93; Domenger and Grimm, Human Molecular Genetics, 28(R1):R3-R14 (October 2019); and WO/2019/200286, and references cited therein.

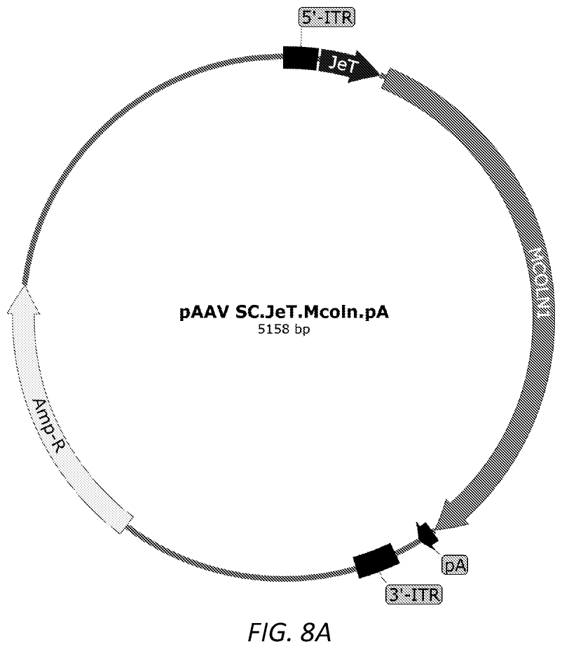

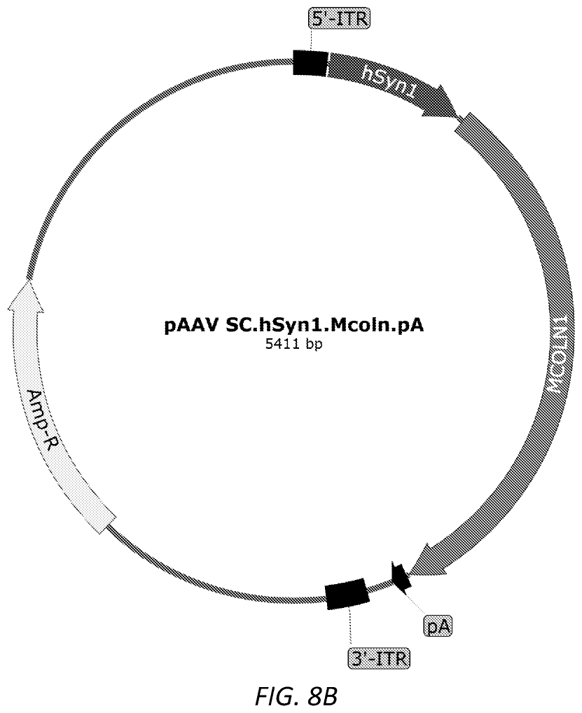

Exemplary vectors are shown in FIGS. 8A-B (for use in humans, the Amp resistance cassette could be deleted and replaced with a kanamycin resistance cassette), and sequences are provided below (SEQ ID NOs:4 and 5).

Also provided herein are compositions and formulations comprising the vectors, e.g., in a sterile carrier. Formulation of pharmaceutically-acceptable excipients and carrier solutions is well-known to those of skill in the art. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the AAV can be formulated for delivery encapsulated in a lipid particle, a liposome, a vesicle, a nanosphere, or a nanoparticle (e.g., conjugated to a nanoparticle) or the like.

Also provided herein are methods of making the AAV, as well as host cells comprising the AAV. Methods for obtaining recombinant AAVs having a desired capsid protein are well known in the art. (See, e.g., US 2003/0138772 and WO 2019/200286, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins.

### EXAMPLES

The invention is further described in the following examples, which do not limit the scope of the invention described in the claims.

Materials and Methods

The following materials and methods were used in the Examples below.

Animals

Mcoln1^{-/-} mice were maintained and genotyped as previously described (1). The Mcoln1^{-/-} breeders for this study were obtained by backcrossing onto a C57Bl6J background for more than 10 generations. Experimental cohorts were obtained from either Mcoln1^{+/-}×Mcoln1^{+/-} or Mcoln1^{+/-}×Mcoln1^{-/-} mating. Mcoln1^{+/-} littermates were used as controls. Experiments were performed according to the Institutional and National Institutes of Health guidelines and approved by the Massachusetts General Hospital Institutional Animal Care and Use Committee. Animals were assigned to the experimental groups in a random order and handling and testing was performed by investigator blinded to treatment group info.

Constructs

Three plasmids were used for MCOLN1 expression: pAAVss-CMV-MCOLN1-FF2A-eGFP, pAAVsc-JeT-MCOLN1 and pAAVsc-SYN1-MCOLN1. The expression vectors were obtained from Dr. Vandenberghe's lab at MEEI. pAAVss-CMV-MCOLN1-FF2A-eGFP had a bGH polyA, and a human MCOLN1 cDNA was synthesized and subcloned (ncbi.nlm.nih.gov/gene/57192). To produce pAAVsc-JeT-MCOLN1 and pAAVsc-SYN1-MCOLN1 we used the original pAAV SC.CMV.EGFP.BGH vector, where the CMV-eGFP-bGHpolyA expression cassette was replaced by human MCOLN1 cDNA with short synthetic polyA (2) and either minimal synthetic JeT (3) or human SYN1 (4) promoters.

Virus Preparation

AAV-PHP.B-CMV-MCOLN1-FF2A-eGFP (referred to as PHP.b-MCOLN1 in the text) and scAAV9-JeT MCOLN1 and scAAV9-SYN1-MCOLN1 viral stocks were prepared at the Gene Transfer Vector Core (vector.meei.harvard.edu) at Massachusetts Eye and Ear Infirmary. AAV preparations were produced by triple-plasmid transfection as described previously (5). Near-confluent monolayers of HEK293 cells were used to perform large scale polyethylenimine transfections of AAVcis, AAVtrans, and adenovirus helper plasmid in a ten-layer hyperflask (Corning, Corning, N.Y.). Downstream purification process, titration and evaluation of purity was performed as described previously (Lock, 2010).

ICV Injections

The viral solution was diluted in sterile saline (0.9% NaCl) with 0.05% trypan blue. Each pup was injected with a maximal injection volume of 5 ul via a 10 ul Hamilton syringe (Hamilton Company, Reno, Nev.) with a 30G needle. Pups were anesthetized on ice for 5 min until loss of pedal withdrawal reflex. The pup was then placed on a fiber optic light source to illuminate ventricles for injection. The injection site was marked with non-toxic laboratory pen about 0.25 mm lateral to sagittal suture and 0.50-0.75 mm rostral to neonatal coronary suture (0.25 mm lateral to confluence of sinuses). Pups were injected with either saline, scAAV9-JeT-MCOLN1 at a dose of 2×10¹⁰ vg/mouse, 1×10¹⁰ vg/mouse or 0.4×10¹⁰ vg/mouse or scAAV9-JeT-MCOLNJ at a dose of 2×10¹⁰ vg/mouse. After, injection the pup was placed under a warming lamp and monitored for 5-10 min for recovery until movement and responsiveness was fully restored.

IV Injections

Mice were restrained in a plexiglass restrainer. The tail veins were dilated in warm water for 1 minute. 30G-needle insulin syringes (Cat. No 328466,BD, San Diego, Calif.) were used with total injection volume of 70-100 ul per mouse. Mice were injected with either saline, AAV-PHP.b-CMV-MCOLN1 at a dose of 1×10¹² vg/mouse or scAAV9-JeT-MCOLN1 at a dose of 5×10¹¹ vg/mouse.

Behavioral Testing

Open field testing was performed on naive male and female mice at either two or four months of age under regular light conditions. Each mouse was placed in the center of a 27×27 cm2 Plexiglas arena, and the horizontal and vertical activity were recorded by the Activity Monitor program (Med Associates Fairfax, Vt.). Data were analyzed during the first 15 minutes in the arena. Zone analysis was performed to measure movements/time spent in the central (8×8 cm²) versus peripheral (residual) zone of the arena. Statistical significance was determined using an unpaired T-test.

Motor coordination and balance was tested in on an accelerating rotarod (Med Associates, VT). Latency to fall from the rotating rod was recorded in 3 trials per day (accelerating speed from 4 to 40 rpm over 5 min) during 2 days of testing. To follow the progression of motor decline, performance of mice was tested monthly starting at 4 months until the motor dysfunction in Mcoln1^{-/-} mice made it impossible to perform the task. Cox-regression and log-rank analysis was used to determine probability of falling from the rod.

RNA Extraction and qPCR Analysis

Mouse tissues were disrupted and homogenized using QIAzol lysis reagent (Qiagen, Hilden, Germany) and the Tissue Lyser instrument (Qiagen, Hilden, Germany). 30 mg of snap frozen tissues were processed adding 1 ml of QIAzol reagent in presence of one 5 mm stainless steel bead (Qiagen, Hilden, Germany). Total RNA isolation from homogenized tissues was performed using Qiagen RNeasy kit (Qiagen, Hilden, Germany) and genomic DNA was eliminated performing DNase (Qiagen, Hilden, Germany) digestion on columns following procedures indicated by the provider. cDNA was generated using High-Capacity cDNA Reverse Transcription kit (Applied Biosystems, Foster City, Calif.) according to manual instruction. cDNA produced from 500 ng of starting RNA was diluted and 40 ng were used to perform qPCR using LightCycler 480 Probes Master mix (Roche Diagnostics, Mannheim, Germany). The real-time PCR reaction was run on LightCycler 480 (Roche Diagnostics, Mannheim, Germany) using TaqMan premade gene expression assays (Applied Biosystems, Foster City, Calif.). Mus musculus: GAPDH (FAM)-Mm99999915_g1, MBP, (FAM)-Mm01266402_m1, GFAP (FAM)-Mm01253033_m1, CD68 (FAM)-Mm03047343_m1; Homo Sapiens Mucolipin-1 (FAM)-Hs01100653_m1. The ΔΔCt method was used to calculate relative gene expression, where Ct corresponds to the cycle threshold. ΔCt values were calculated as the difference between Ct values from the target gene and the housekeeping gene GAPDH.

Tissue Collection and Processing

Mice were sacrificed using a carbon dioxide chamber. Immediately after euthanasia, mice were transcardially perfused with ice-cold phosphate buffered saline (PBS). After bisecting the brain across the midline, half was used to isolate cerebellum and cortex. The other half was post-fixed in 4% paraformaldehyde in PBS for 48 h, washed with PBS, cryoprotected in 30% sucrose in PBS for 24 h, frozen in isopentane and stored at −80° C. In addition to brain tissue, liver, muscle and sciatic nerve tissue were collected and snap-frozen over dry ice before storage at −80° C.

Immunohistochemistry, Imaging, and Analysis

For histological analysis, 40 μm coronal sections were cut using a cryostat (Leica Microsystems, Wetzlar, Germany) and collected into 96 well plates containing cryoprotectant (TBS, 30% ethylene glycol, 15% sucrose). These sections were stored at 4° C. prior or frozen at −80° C. Prior to staining for LAMP1 and Myelin Basic Protein (MBP), samples were randomized and coded to create blinded conditions for analysis. Staining of free-floating sections was done in a 96 well plate. Sections were blocked in 0.1% Triton X-100, 10% normal horse serum (NHS), 2% bovine serum albumin (BSA), and 1% glycine in PBS and incubated with primary antibodies diluted in antibody buffer: 10% NHS and 2% BSA overnight. The following primary antibodies were used: a-LAMP1 antibody (Rat 1:1000, BD San Diego, Calif., Cat ID: 553792); MBP (Mouse, 1:1000, Millipore Sigma, Jaffrey, NH, NE1019). Sections were washed in PBS-Tween (0.05%) at room temperature then incubated with secondary antibodies in antibody buffers for 1 hour at room temperature. The following secondary antibodies were used: goat-anti-rat AlexaFluor 546 (1:500; Invitrogen, Eugene, OR), goat-anti-mouse AlexaFluor 555 (1:500; Invitrogen, Eugene, Oreg.). Sections were counterstained with NucBlue nuclear stain (Life Technologies, Eugene, Oreg.). After staining, sections were mounted onto glass SuperFrost plus slides (Fisher Scientific, Pittsburgh, Pa.) with Immu-Mount (Fisher Scientific, Pittsburgh, Pa.).

Images were acquired on DM8i Leica Inverted Epifluorescence Microscope with Adaptive Focus (Leica Microsystems, Buffalo Grove, Ill.) with Hamamatsu Flash 4.0 camera and advanced acquisition software package MetaMorph 4.2 (Molecular Devices, LLC, San Jose, Calif.) using an automated stitching function. The exposure time was kept constant for all sections within the same IHC experiment. Image analysis was performed using Fiji software (NIH, Bethesda, Md.). For corpus callosum area measurements areas of interest were selected on MBP images in each section and normalized to the whole section area. For LAMP1 images particle analysis and % of area measurements were made after same thresholding settings were allied to all images. All images were decoded after the measurements were taken. Area and mean pixel intensity values were averaged per genotype/treatment group and compared between genotypes using unpaired T-test.

Statistical Analysis

Data presented as mean values and SEM or median values and interquartile range. Statistical analysis was performed in GraphPad Prism 7.04 software (GraphPad, La Jolla, Calif.) using either unpaired T-test, two-way ANOVA or log-rank as tests detailed in specific subsections of Methods. P values were indicated as follows throughout the manuscript: n.s.=p<0.05; * p=<0.05; ** p=<0.01, *** p=<0.001,**** p=<0.0001.

Exemplary Viral Sequences:

>pAAV SC.JeT.Mcoln.pA (5158 bp)
(SEQ ID NO: 4)
CTGCGCGCTCGCTCGCTCACTGAGGCCGCCCGGGCAAAGCCCGGGCGTCGGGCGACCTT
TGGTCGCCCGGCCTCAGTGAGCGAGCGAGCGCGCAGAGAGGGAGTGGTTAAGCTAGCGA
ATTCGGGCGGAGTTAGGGCGGAGCCAATCAGCGTGCGCCGTTCCGAAAGTTGCCTTTTA
TGGCTGGGCGGAGAATGGGCGGTGAACGCCGATGATTATATAAGGACGCGCCGGGTGTG
GCACAGCTAGTTCCGTCGCAGCCGGGATTTGGGTCGCGGTTCTTGTTTGTGGATCCCTG
TGATCGTCACTTGACAGTGTCCAGGCGGCCGCCACCATGACAGCCCCGGCGGGTCCGCG
CGGCTCAGAGACCGAGCGGCTTCTGACCCCCAACCCCGGGTATGGGACCCAGGCGGGGC
CTTCACCGGCCCCTCCGACACCCCCAGAAGAGGAAGACCTTCGCCGTCGTCTCAAATAC
TTTTTCATGAGTCCCTGCGACAAGTTTCGAGCCAAGGGCCGCAAGCCCTGCAAGCTGAT
GCTGCAAGTGGTCAAGATCCTGGTGGTCACGGTGCAGCTCATCCTGTTTGGGCTCAGTA
ATCAGCTGGCTGTGACATTCCGGGAAGAGAACACCATCGCCTTCCGACACCTCTTCCTG
CTGGGCTACTCGGACGGAGCGGATGACACCTTCGCAGCCTACACGCGGGAGCAGCTGTA
CCAGGCCATCTTCCATGCTGTGGACCAGTACCTGGCGTTGCCTGACGTGTCACTGGGCC
GGTATGCGTATGTCCGTGGTGGGGGTGACCCTTGGACCAATGGCTCAGGGCTTGCTCTC
TGCCAGCGGTACTACCACCGAGGCCACGTGGACCCGGCCAACGACACATTTGACATTGA
TCCGATGGTGGTTACTGACTGCATCCAGGTGGATCCCCCCGAGCGGCCCCCTCCGCCCC
CCAGCGACGATCTCACCCTCTTGGAAAGCAGCTCCAGTTACAAGAACCTCACGCTCAAA
TTCCACAAGCTGGTCAATGTCACCATCCACTTCCGGCTGAAGACCATTAACCTCCAGAG
CCTCATCAATAATGAGATCCCGGACTGCTATACCTTCAGCGTCCTGATCACGTTTGACA
ACAAAGCACACAGTGGGCGGATCCCCATCAGCCTGGAGACCCAGGCCCACATCCAGGAG
TGTAAGCACCCCAGTGTCTTCCAGCACGGAGACAACAGCTTCCGGCTCCTGTTTGACGT
GGTGGTCATCCTCACCTGCTCCCTGTCCTTCCTCCTCTGCGCCCGCTCACTCCTTCGAG
GCTTCCTGCTGCAGAACGAGTTTGTGGGGTTCATGTGGCGGCAGCGGGGACGGGTCATC
AGCCTGTGGGAGCGGCTGGAATTTGTCAATGGCTGGTACATCCTGCTCGTCACCAGCGA
TGTGCTCACCATCTCGGGCACCATCATGAAGATCGGCATCGAGGCCAAGAACTTGGCGA
GCTACGACGTCTGCAGCATCCTCCTGGGCACCTCGACGCTGCTGGTGTGGGTGGGCGTG
ATCCGCTACCTGACCTTCTTCCACAACTACAATATCCTCATCGCCACACTGCGGGTGGC
CCTGCCCAGCGTCATGCGCTTCTGCTGCTGCGTGGCTGTCATCTACCTGGGCTACTGCT
TCTGTGGCTGGATCGTGCTGGGGCCCTATCATGTGAAGTTCCGCTCACTCTCCATGGTG
TCTGAGTGCCTGTTCTCGCTCATCAATGGGGACGACATGTTTGTGACGTTCGCCGCCAT
GCAGGCGCAGCAGGGCCGCAGCAGCCTGGTGTGGCTCTTCTCCCAGCTCTACCTTTACT
CCTTCATCAGCCTCTTCATCTACATGGTGCTCAGCCTCTTCATCGCGCTCATCACCGGC
GCCTACGACACCATCAAGCATCCCGGCGGCGCAGGCGCAGAGGAGAGCGAGCTGCAGGC
CTACATCGCACAGTGCCAGGACAGCCCCACCTCCGGCAAGTTCCGCCGCGGGAGCGGCT
CGGCCTGCAGCCTTCTCTGCTGCTGCGGAAGGGACCCCTCGGAGGAGCATTCGCTGCTG
GTGAATTAATAAGCTTAATAAAAGATCTTTATTTTCATTAGATCTGTGTGTTGGTTTTT
TGTGTGCTCGAGTTAAGGGCGAATTCCCGATAAGGATCTTCCTAGAGCATGGCTACGTA
GATAAGTAGCATGGCGGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCCTTAATTAACCT
AATTCACTGGCCGTCGTTTTACAACGTCGTGACTGGGAAAACCCTGGCGTTACCCAACT
TAATCGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGGACGCGCCCTGTAGC
GGCGCATTAAGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCATCGCCCTG
ATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATT
TTGCCGATTTCGGCCTATTGGTTAAAAAATGAGCTGATTTAACAAAAATTTAACGCGAA
TTTTAACAAAATATTAACGTTTATAATTTCAGGTGGCATCTTTCGGGGAAATGTGCGCG
GAACCCCTATTTGTTTATTTTTCTAAATACATTCAAATATGTATCCGCTCATGAGACAA
TAACCCTGATAAATGOTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATC
GAACTGGATCTCAATAGTGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCG
GGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGC
CATAACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGT
AATGGTAACAACGTTGCGCAAACTATTAACTGGCGAACTACTTACTCTAGCTTCCCGGC
AACAATTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTATCTACACGA
CGGGGAGTCAGGCAACTATGGATGAACGAAATAGACAGATCGCTGAGATAGGTGCCTCA
CTGATTAAGCATTGGTAACTGTCAGACCAAGTTTACTCATATATACTTTAGATTGATTT
AAAACTTCATTTTTAATTTAAAAGGATCTAGGTGAAGATCCTTTTTGATAATCTCATGA
CCAAAATCCCTTAACGTGAGTTTTCGTTCCACTGAGCGTCAGACCCCGTAGAAAAGATC
AAAGGATCTTCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCGTAG
TTAGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGCGGTT
TTGCTCACATGTTCTTTCCTGCGTTATCCCCTGATTCTGTGGATAACCGTATTACCGCC
TTTGAGTGAGCTGATACCGCTCGCCGCAGCCGAACGACCGAGCGCAGCGAGTCAGTGAG
CGAGGAAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCAACGCA
ATTAATGTGAGTTAGCTCACTCATTAGGCACCCCAGGCTTTACACTTTATGCTTCCGGC
TCGTATGTTGTGTGGAATTGTGAGCGGATAACAATTTCACACAGGAAACAGCTATGACC
ATGATTACGCCAGATTTAATTAAGG
>pAAV SC.hSyn1.Mcoln.pA (5411 bp)
(SEQ ID NO: 5)
CTGCGCGCTCGCTCGCTCACTGAGGCCGCCCGGGCAAAGCCCGGGCGTCGGGCGACCTT
TGGTCGCCCGGCCTCAGTGAGCGAGCGAGCGCGCAGAGAGGGAGTGGTTAAGCTAGCAG
TGCAAGTGGGTTTTAGGACCAGGATGAGGCGGGGTGGGGGTGCCTACCTGACGACCGAC
CCCGACCCACTGGACAAGCACCCAACCCCCATTCCCCAAATTGCGCATCCCCTATCAGA
GAGGGGGAGGGGAAACAGGATGCGGCGAGGCGCGTGCGCACTGCCAGCTTCAGCACCGC
GGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGT
CCGCGCCGCCGCCGGCCCAGCCGGACCGCACCACGCGAGGCGCGAGATAGGGGGGCACG
GGCGCGACCATCTGCGCTGCGGCGCCGGCGACTCAGCGCTGCCTCAGTCTGCGGTGGGC
AGCGGAGGAGTCGTGTCGTGCCTGAGAGCGCAGGTGTCCAGGCGGCCGCCACCATGACA
GCCCCGGCGGGTCCGCGCGGCTCAGAGACCGAGCGGCTTCTGACCCCCAACCCCGGGTA
TGGGACCCAGGCGGGGCCTTCACCGGCCCCTCCGACACCCCCAGAAGAGGAAGACCTTC
GCCGTCGTCTCAAATACTTTTTCATGAGTCCCTGCGACAAGTTTCGAGCCAAGGGCCGC
AAGCCCTGCAAGCTGATGCTGCAAGTGGTCAAGATCCTGGTGGTCACGGTGCAGCTCAT
CCTGTTTGGGCTCAGTAATCAGCTGGCTGTGACATTCCGGGAAGAGAACACCATCGCCT
TCCGACACCTCTTCCTGCTGGGCTACTCGGACGGAGCGGATGACACCTTCGCAGCCTAC
ACGCGGGAGCAGCTGTACCAGGCCATCTTCCATGCTGTGGACCAGTACCTGGCGTTGCC
TGACGTGTCACTGGGCCGGTATGCGTATGTCCGTGGTGGGGGTGACCCTTGGACCAATG
GCTCAGGGCTTGCTCTCTGCCAGCGGTACTACCACCGAGGCCACGTGGACCCGGCCAAC
GACACATTTGACATTGATCCGATGGTGGTTACTGACTGCATCCAGGTGGATCCCCCCGA
GCGGCCCCCTCCGCCCCCCAGCGACGATCTCACCCTCTTGGAAAGCAGCTCCAGTTACA
AGAACCTCACGCTCAAATTCCACAAGCTGGTCAATGTCACCATCCACTTCCGGCTGAAG
ACCATTAACCTCCAGAGCCTCATCAATAATGAGATCCCGGACTGCTATACCTTCAGCGT
CCTGATCACGTTTGACAACAAAGCACACAGTGGGCGGATCCCCATCAGCCTGGAGACCC
AGGCCCACATCCAGGAGTGTAAGCACCCCAGTGTCTTCCAGCACGGAGACAACAGCTTC
CGGCTCCTGTTTGACGTGGTGGTCATCCTCACCTGCTCCCTGTCCTTCCTCCTCTGCGC
CCGCTCACTCCTTCGAGGCTTCCTGCTGCAGAACGAGTTTGTGGGGTTCATGTGGCGGC
AGCGGGGACGGGTCATCAGCCTGTGGGAGCGGCTGGAATTTGTCAATGGCTGGTACATC
CTGCTCGTCACCAGCGATGTGCTCACCATCTCGGGCACCATCATGAAGATCGGCATCGA
GGCCAAGAACTTGGCGAGCTACGACGTCTGCAGCATCCTCCTGGGCACCTCGACGCTGC
TGGTGTGGGTGGGCGTGATCCGCTACCTGACCTTCTTCCACAACTACAATATCCTCATC
GCCACACTGCGGGTGGCCCTGCCCAGCGTCATGCGCTTCTGCTGCTGCGTGGCTGTCAT
CTACCTGGGCTACTGCTTCTGTGGCTGGATCGTGCTGGGGCCCTATCATGTGAAGTTCC
GCTCACTCTCCATGGTGTCTGAGTGCCTGTTCTCGCTCATCAATGGGGACGACATGTTT
GTGACGTTCGCCGCCATGCAGGCGCAGCAGGGCCGCAGCAGCCTGGTGTGGCTCTTCTC
CCAGCTCTACCTTTACTCCTTCATCAGCCTCTTCATCTACATGGTGCTCAGCCTCTTCA
TCGCGCTCATCACCGGCGCCTACGACACCATCAAGCATCCCGGCGGCGCAGGCGCAGAG
GAGAGCGAGCTGCAGGCCTACATCGCACAGTGCCAGGACAGCCCCACCTCCGGCAAGTT
CCGCCGCGGGAGCGGCTCGGCCTGCAGCCTTCTCTGCTGCTGCGGAAGGGACCCCTCGG
AGGAGCATTCGCTGCTGGTGAATTAATAAGCTTAATAAAAGATCTTTATTTTCATTAGA
TCTGTGTGTTGGTTTTTTGTGTGCTCGAGTTAAGGGCGAATTCCCGATAAGGATCTTCC
TAGAGCATGGCTACGTAGATAAGTAGCATGGCGGGTTAATCATTAACTACAAGGAACCC
CTAGTGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCGAGCGC
GCAGCCTTAATTAACCTAATTCACTGGCCGTCGTTTTACAACGTCGTGACTGGGAAAAC
CCTGGCGTTACCCAACTTAATCGCCTTGCAGCACATCCCCCTTTCGCCAGCTGGCGTAA
TAGCGAAGAGGCCCGCACCGATCGCCCTTCCCAACAGTTGCGCAGCCTGAATGGCGAAT
GGGACGCGCCCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCCTTCCTTTCT
CGCCACGTTCGCCGGCTTTCCCCGTCAAGCTCTAAATCGGGGGCTCCCTTTAGGGTTCC
GATTTAGTGCTTTACGGCACCTCGACCCCAAAAAACTTGATTAGGGTGATGGTTCACGT
AGTGGGCCATCGCCCTGATAGACGGTTTTTCGCCCTTTGACGTTGGAGTCCACGTTCTT
TAATAGTGGACTCTTGTTCCAAACTGGAACAACACTCAACCCTATCTCGGTCTATTCTT
TTGATTTATAAGGGATTTTGCCGATTTCGGCCTATTGGTTAAAAAATGAGCTGATTTAA
CAAAAATTTAACGCGAATTTTAACAAAATATTAACGTTTATAATTTCAGGTGGCATCTT
TCGGGGAAATGTGCGCGGAACCCCTATTTGTTTATTTTTCTAAATACATTCAAATATGT
ATCCGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGGTG
CACGAGTGGGTTACATCGAACTGGATCTCAATAGTGGTAAGATCCTTGAGAGTTTTCGC
CCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGAC
AACGATCGGAGGACCGAAGGAGCTAACCGCTTTTTTGCACAACATGGGGGATCATGTAA
CTCGCCTTGATCGTTGGGAACCGGAGCTGAATGAAGCCATACCAAACGACGAGCGTGAC
ACCACGATGCCTGTAGTAATGGTAACAACGTTGCGCAAACTATTAACTGGCGAACTACT
TACTCTAGCTTCCCGGCAACAATTAATAGACTGGATGGAGGCGGATAAAGTTGCAGGAC
CACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTAT
CGTAGTTATCTACACGACGGGGAGTCAGGCAACTATGGATGAACGAAATAGACAGATCG
CTGAGATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGATCCT
TTTTGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGCCTACATAC
CTCGCTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTTTTACGGTTCCT
GGCCTTTTGCTGCGGTTTTGCTCACATGTTCTTTCCTGCGTTATCCCCTGATTCTGTGG
ATAACCGTATTACCGCCTTTGAGTGAGCTGATACCGCTCGCCGCAGCCGAACGACCGAG
CGCAGCGAGTCAGTGAGCGAGGAAGCGGAAGAGCGCCCAATACGCAAACCGCCTCTCCC
CGCGCGTTGGCCGATTCATTAATGCAGCTGGCACGACAGGTTTCCCGACTGGAAAGCGG
GCAGTGAGCGCAACGCAATTAATGTGAGTTAGCTCACTCATTAGGCACCCCAGGCTTTA
CACTTTATGCTTCCGGCTCGTATGTTGTGTGGAATTGTGAGCGGATAACAATTTCACAC
AGGAAACAGCTATGACCATGATTACGCCAGATTTAATTAAGG

### Example 1. MCOLN1 Gene Transfer in Juvenile Pre-Symptomatic Mcoln1^{-/-} Mice Prevents Onset of Motor Deficits

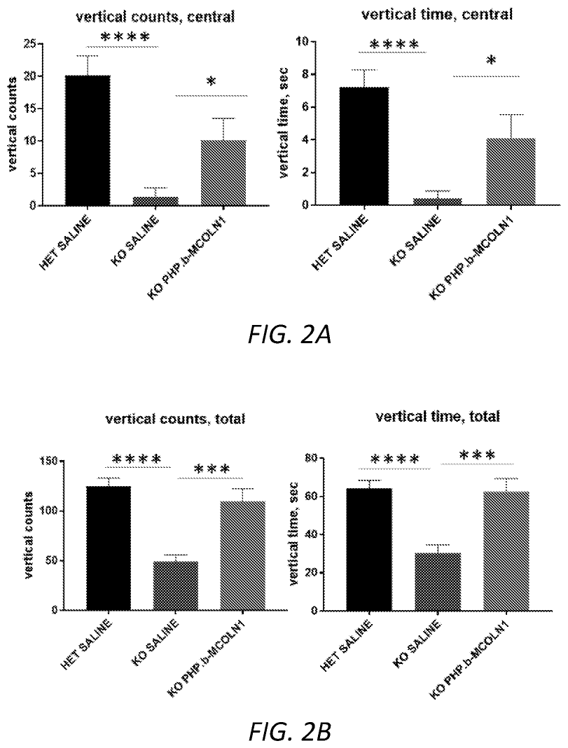

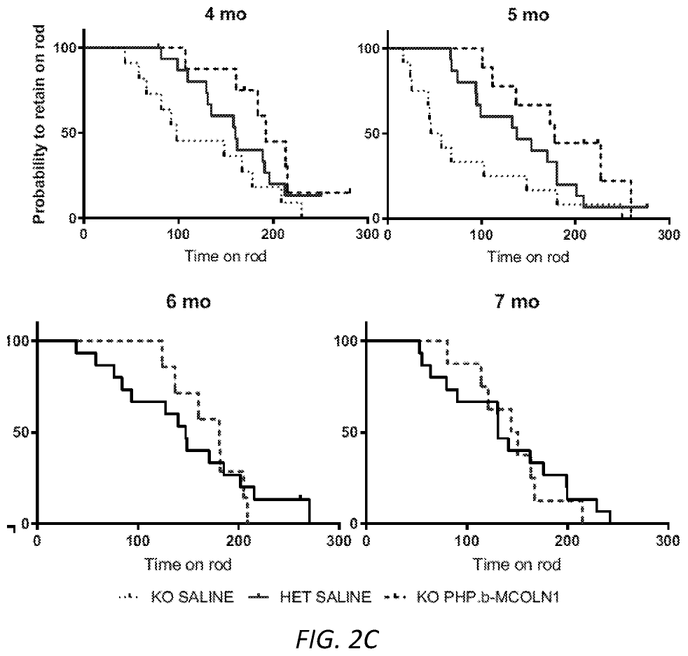

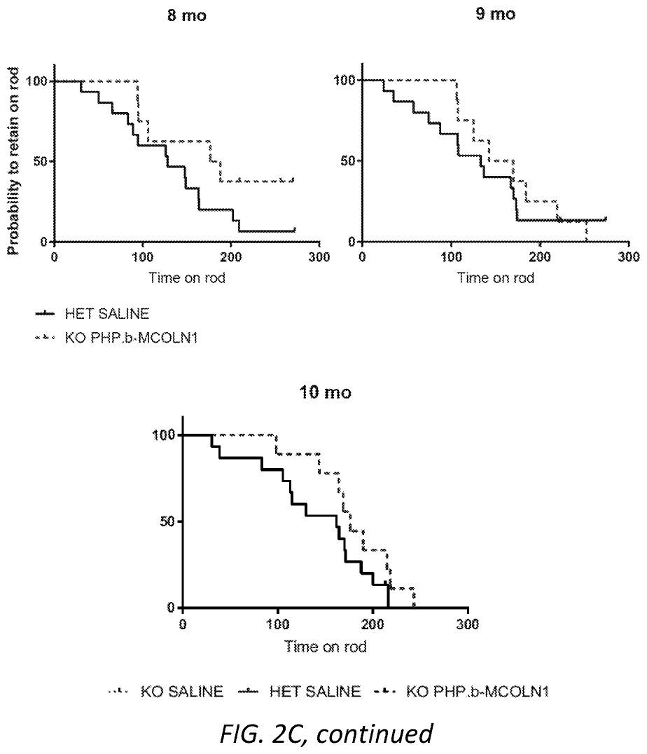

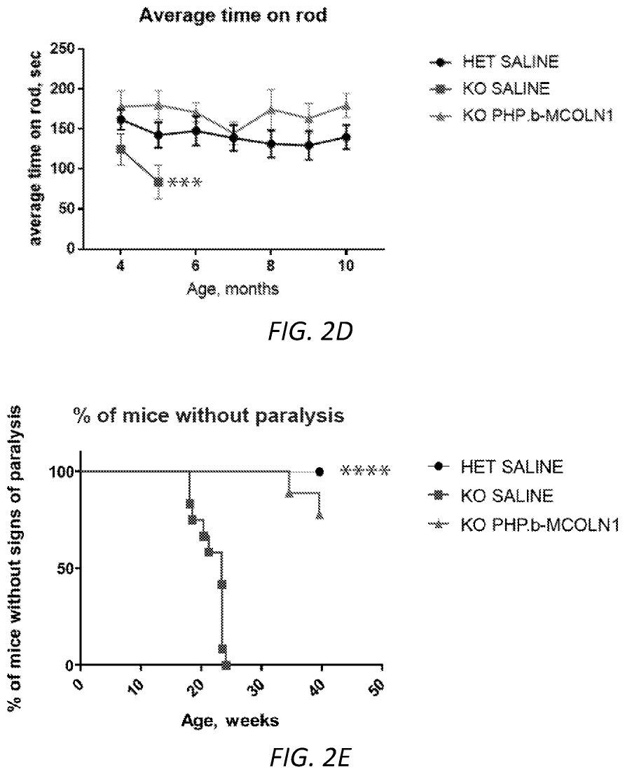

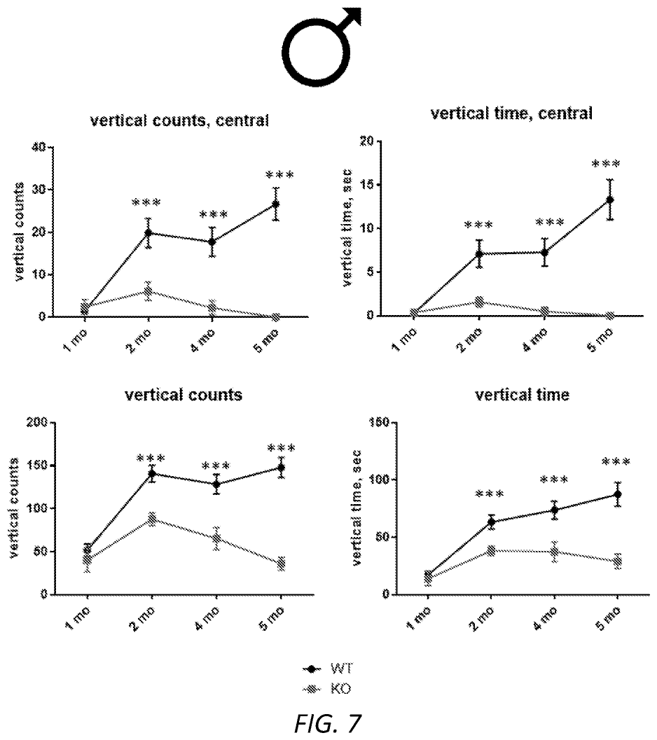

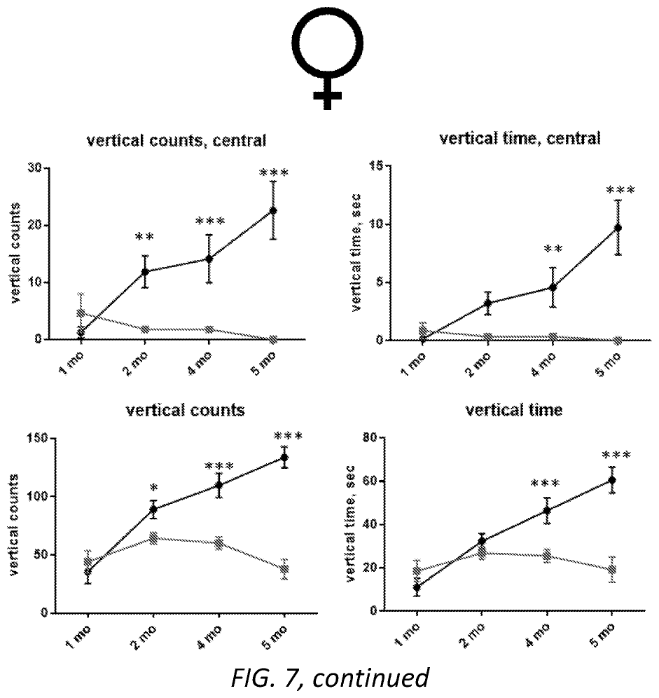

Mcoln1^{-/-} mice mimic all the main manifestations of the human mucolipidosis IV disease, including neurologic deficits and brain pathology, gastric pathology and high systemic gastrin, and eye pathology, with exception to corneal clouding (12). The earliest motor deficits appear in the form of reduced vertical activity in the open field test at the age of two months (FIG. 7). Motor dysfunction in Mcoln1^{-/-} mice progresses in the course of disease resulting in decreased performance in the rotarod test at the age of four months (FIGS. 2 C,D and 6 D, E), significant gait deficits (6) and, eventually, hind limps paralysis and pre-mature death at around 7-8 months of age (6).

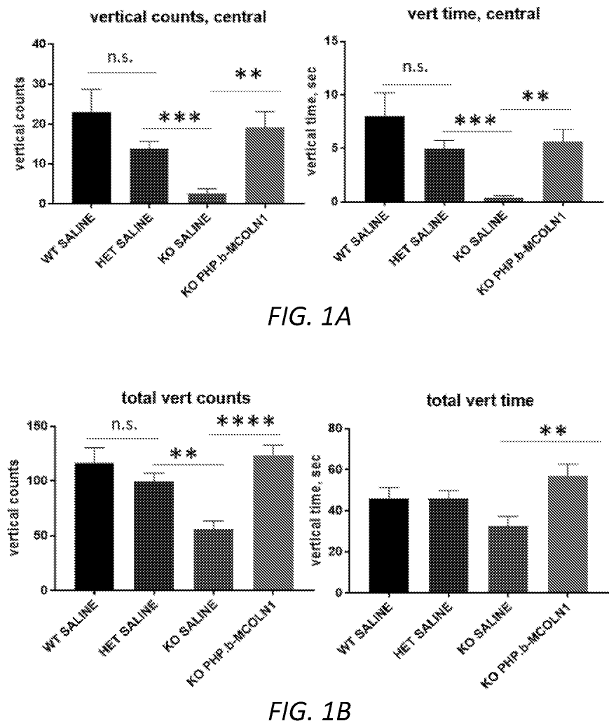

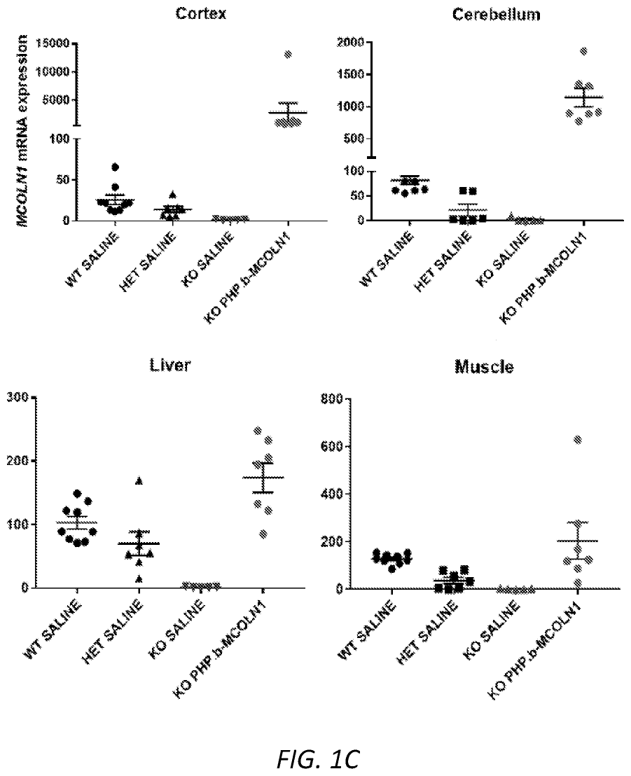

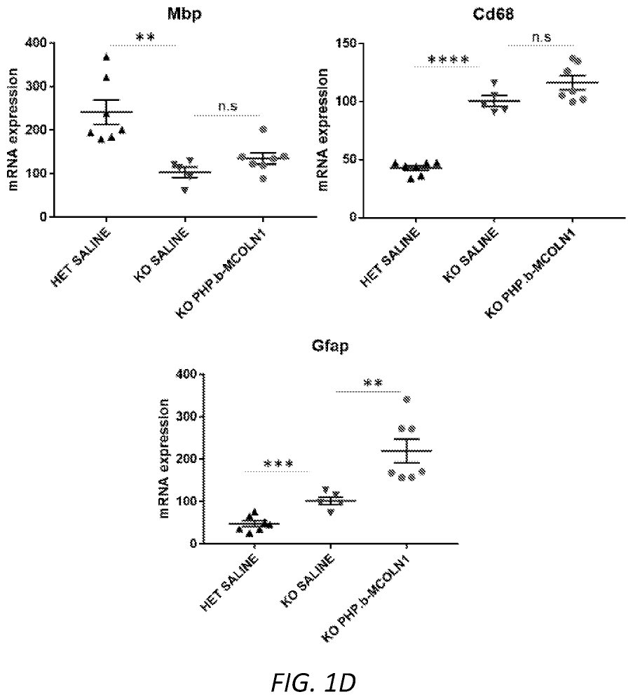

Here, we aimed to test whether CNS-targeted transfer of the human MCOLN1 gene in the juvenile KO mice prior to the first measurable symptom onset at the age of 5-6 weeks will be able to prevent motor deficits at the age of two months. We administered Mcoln1^{-/-} and control Mcoln1^{-/-} male mice either saline or AAV-PHP.b-CMV-MCOLN1-F[urin]F2a-eGFP (a.k.a PHP.b-MCOLN1) intravenously at the age of 5-6 weeks after they reached a body mass of 19 g. Four weeks after injections, at the age of 2 months, mice were tested in the open field arena and euthanized for tissue collection (Table 1). The open field test showed complete prevention of vertical activity decline in the PHP.b-MCOLN1-treated Mcoln1^{-/-} mice (FIGS. 1A, B). Post-mortem tissue analysis showed high expression of the human MCOLN1 transgene in the brain parenchyma, particularly, in the cerebral cortex and cerebellum, and detectable expression in peripheral tissues such as liver and muscle (FIG. 1C). Of note, the human-specific MCOLN1 Taq-man assay that we used for transcriptional analysis (Hs01100653_m1, Applied Biosystems) has 87-94% homology with the mouse Mcoln1 sequence and detects endogenous murine Mcoln1 transcripts in a dose-specific manner as demonstrated on the graphs in FIG. 1C for Mcoln1^{-/-}, Mcoln1^{-/-} and Mcoln1^{-/-} saline-treated samples.

The major histopathological hallmarks of MLIV in the Mcoln1^{-/-} mouse brain at the age of two months are reduced brain myelination, microgliosis and astrocytosis (1, 7-9). Despite the observed strong effect of the MCOLN1 gene transfer on motor function in this cohort (FIG. 1A), qRT-PCR analysis of the post-mortem brain tissue showed no changes in brain myelination, as shown by expression of the myelination marker Mbp, or on activation of microglia, as demonstrated by increased expression of microglial activation marker Cd68 (FIG. 1D). Furthermore, we observed elevated expression of the astrocyte activation marker Gfap in the PHP.b-MCOLN1-treated as compared to saline-treated Mcoln1^{-/-} mice, which may reflect response either to the viral vector or to the expression of a non-self protein in the Mcoln1^{-/-} brain. Importantly, we observed no overt health concerns with this vector dose during all 4 weeks of the trial following vector administration.

TABLE 1
All vector and trial summary.

| | | | Route of | | Age at | Age at |
|---|---|---|---|---|---|---|
| Cohort | Treatment group | N | administration | Dose | treatment | takedown |
| 1 | WT-SALINE | 9 | IV | 1e12 | 5-6 weeks | 9-10 weeks |
| | HET-SALINE | 18 | | vg/mouse | (pre- | |
| | KO-SALINE | 9 | | | symptomatic) | |
| | KO-AAV-PHP.b- | 14 | | | | |
| | MCOLN1 | | | | | |
| 2 | HET-SALINE | 15 | IV | 1e12 | 2 months | 11 months |
| | KO-SALINE | 12 | | vg/mouse | (symptomatic) | untreated |
| | KO-AAV-PHP.b- | 9 | | | | KO) |
| | MCOLN1 | | | | | (around 7 |
| | | | | | | months for |
| 3 | HET-SALINE | 20 | ICV | | P1 (neonatal) | 9-10 weeks |
| | KO-SALINE | 10 | | | | |
| | KO-scAAV9-JeT- | 7 | | 2e10 | | |
| | MCOLN1 | | | vg/mouse | | |
| | KO-scAAV9-JeT- | 16 | | 1e10 | | |
| | MCOLN1 | | | vg/mouse | | |
| | KO-scAAV9-JeT- | 10 | | 4e9 | | |
| | MCOLN1 | | | vg/mouse | | |
| | KO-scAAV9- | 10 | | 2e10 | | |
| | SYN1-MCOLN1 | | | vg/mouse | | |
| 4 | HET- SALINE | 16 | IV | 5e11 | 2 months | 7 months |
| | KO-SALINE | 10 | | vg/mouse | (symptomatic) | |
| | KO-scAAV9-JeT- | 9 | | | | |
| | MCOLN1 | | | | | |

### Example 2. MCOLN1 Gene Transfer in Symptomatic Mcoln1^{-/-} Mice Restores Motor Function and Delays Onset of Paralysis

In all MLIV patients, except for a few milder cases with some residual activity of mucolipin-1, motor deficits manifest very early during the first few months of life. Taking this into account, rescue of the existing motor deficits rather than delay of motor dysfunction onset may be a desirable outcome in a future MLIV clinical trial. To more closely mimic clinical trial design, we set out to test whether administration of the same vector after symptom onset will be able to restore normal motor function. In this experiment Mcoln1^{-/-} and control Mcoln1^{+/-} male mice were intravenously administered either saline or PHP.b-MCOLN1 at the age of two months (Table 1). Motor function was assessed in the open field test once at the age of 4 months. After that, mice were tested in the rotarod test monthly until the completion of the trial. Open field tests revealed significant rescue of vertical activity in the PHP.b-MCOLN1-treated Mcoln1^{-/-} mice in the central zone of the arena (FIG. 2A) and full rescue of vertical activity when quantified in the whole area (FIG. 2B), demonstrating for the first time that MCOLN1 gene replacement can restore neurologic function after it has already been compromised. In the rotarod test saline-treated Mcoln1^{-/-} mice show higher probability to fall off the rod at the age of 4 and 5 months (FIG. 2C) and lower average latency to fall (FIG. 2D). At the age of 6 months saline-treated Mcoln1^{-/-} mice developed profound hind limb weakness and were excluded from testing. By 7 months of age, they developed hind-limb paralysis and had to be euthanized according to humane criteria (FIG. 2E). Remarkably, PHP.b-MCOLN1-treated Mcoln1^{-/-} mice were indistinguishable from the control healthy littermates in the rotarod test until the end of the trial, when they reached 11 months of age (FIGS. 2C, D), and showed significantly delayed time to paralysis. Only two out of nine PHP.b-MCOLN1-treated Mcoln1^{-/-} showed any signs of paralysis after 8 months of age and in average survived the saline Mcoln1^{-/-} group by over 4 months of age (FIG. 2E) showing no signs of neurologic function decline at the time of euthanasia at the age of 11 months of age.

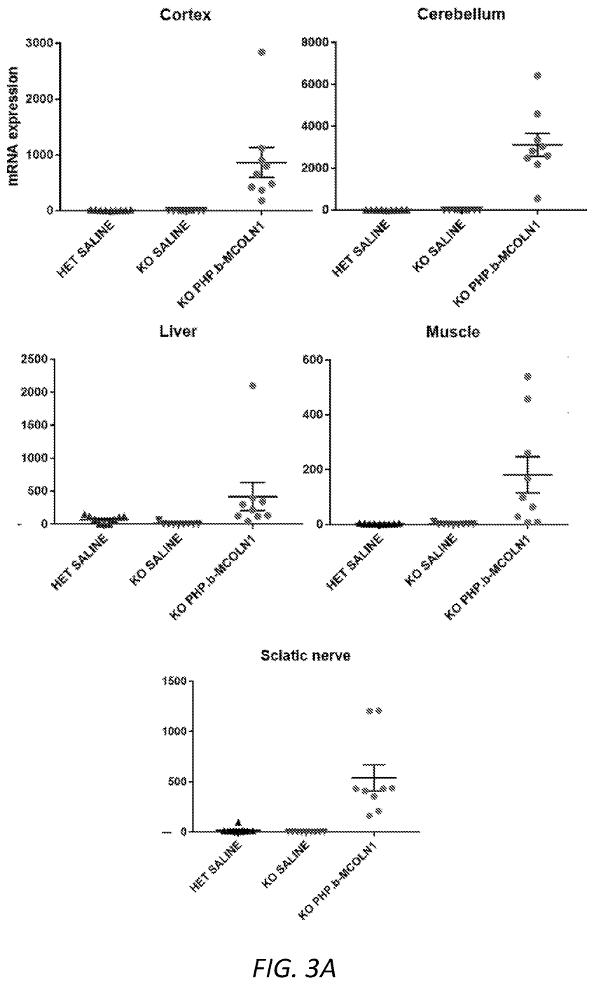

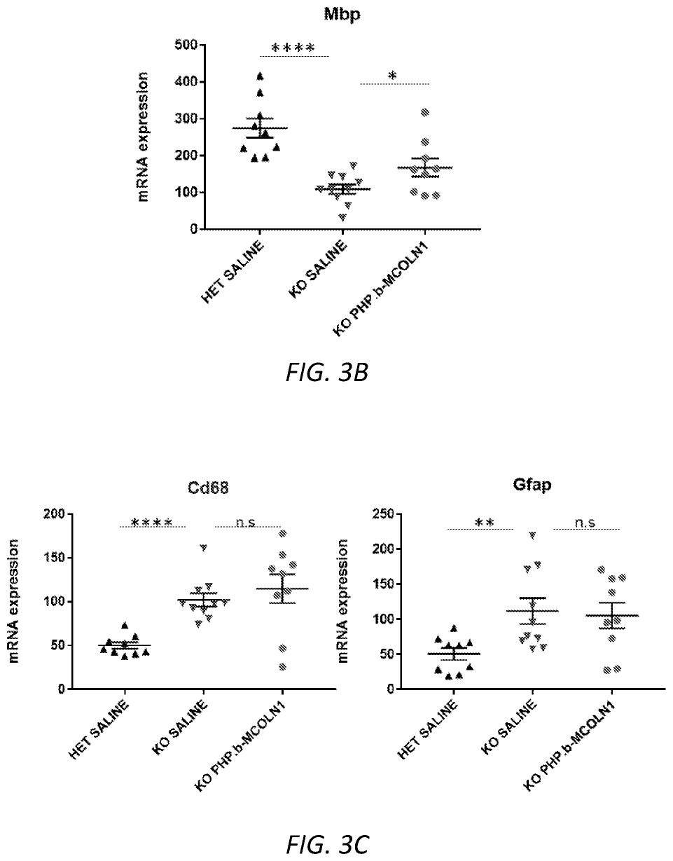

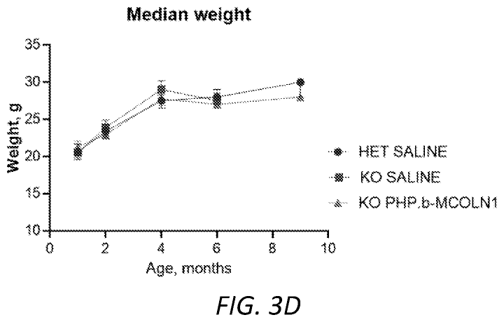

qPCR analysis showed high long-term (tissues were collected 9 months after vector administration) human MCOLN1 transgene expression in the brain tissue, more specifically, in the cerebral cortex and cerebellum, and in peripheral organs, such as liver and muscle. Interestingly, we also saw high MCOLN1 transgene expression in the sciatic nerves, indicating transduction of the PNS (FIG. 3A).

Administration of the PHP.b-MCOLN1 vector to Mcoln1^{-/-} mice at two months of age resulted in partial but statistically significant rescue of brain myelination marker Mbp in the cerebral cortex demonstrating for the first time that myelination deficits in MLIV can be rescued even after the developmental course of brain myelination is completed.

Notably, despite robust rescue of the motor function and high expression in the brain tissue, we observed no changes in the expression of the astrocytosis and microgliosis markers, Gfap and Cd68, in the cerebral cortex of PHP.b-MCOLN1-treated vs. saline-treated Mcoln1^{-/-} mice (FIGS. 3B-D).

### Example 3. Intracerebroventricular Administration of Self-Complementary AAV9-JeT-MCOLN1 is Efficacious in the Mcoln1^{-/-} Mouse Model of MLIV

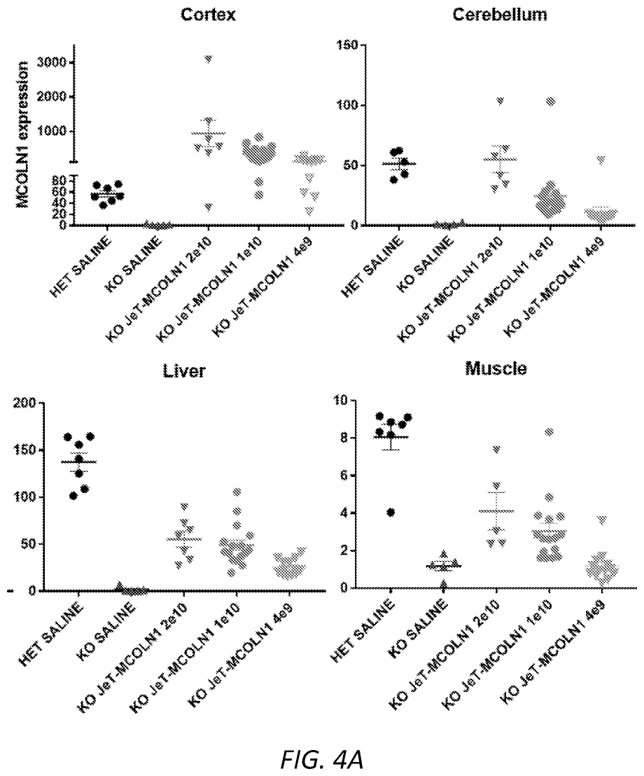

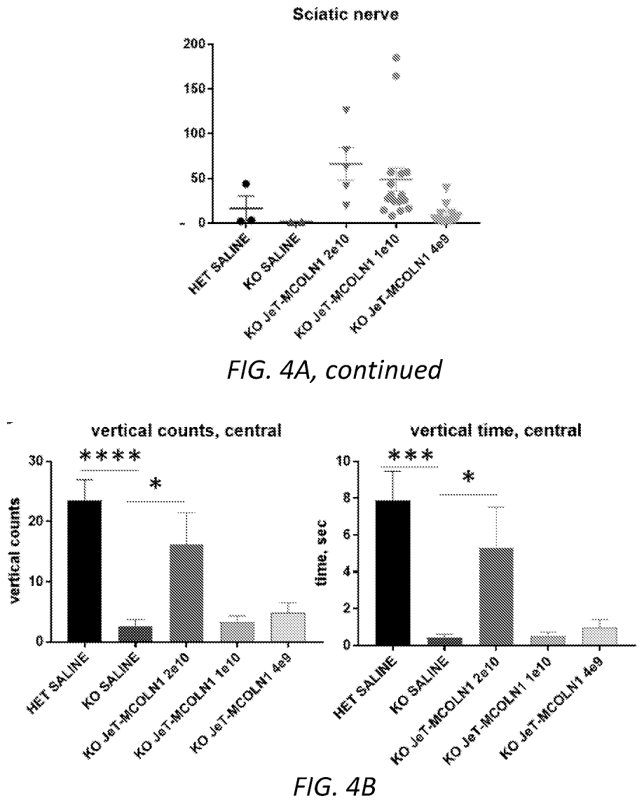

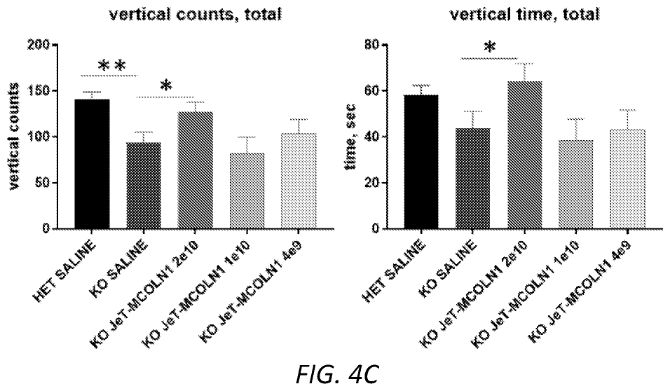

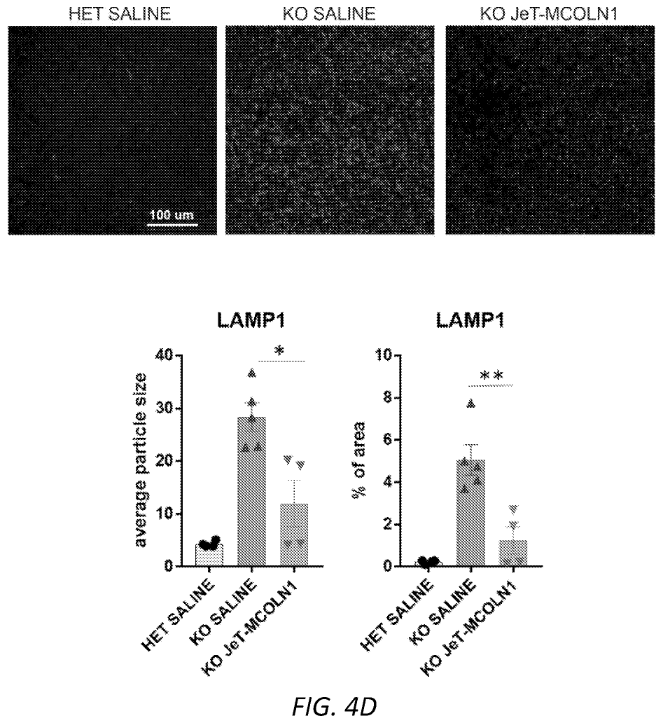

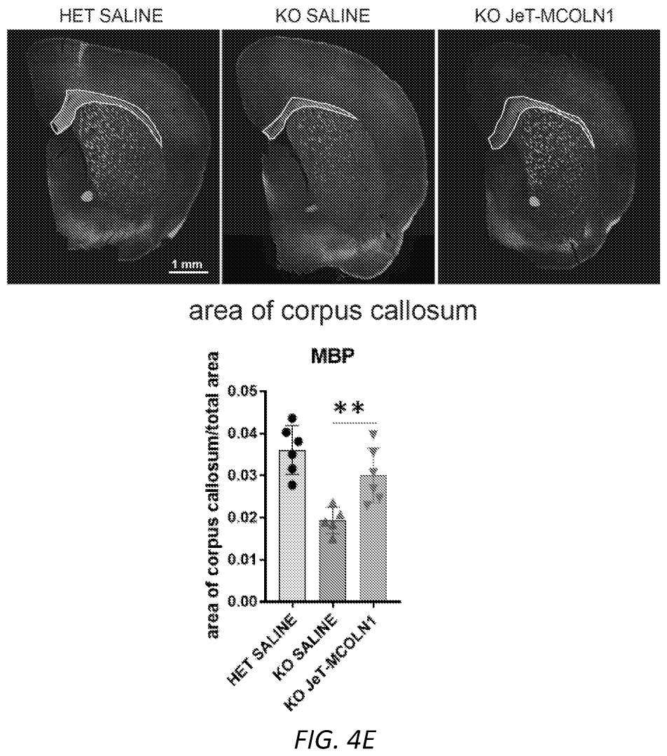

While AAV-PHP.b shows high brain transduction with systemic administration, its ability to penetrate the blood-brain barrier is species-specific with the highest transduction rate in the C57Bl6 mice and a very low brain transduction in non-human primates (NHP) (10, 11). Therefore, we designed an MCOLN1 expression vector suitable for gene transfer in humans based on the clinically proven self-complimentary AAV9 vector (12-14). To drive expression of MCOLN1 we selected a minimal synthetic promoter JeT, which is currently being used in a clinical trial of GAN gene transfer for giant axonal neuropathy (3, 12). For the most efficient and broad targeting of the CNS with scAAV9, we performed intracerebroventicular (ICV) injections in newborn mice at postnatal day 1. Three doses of the vector, 2×10¹⁰, 1×10¹⁰, and 4×10⁹ vg/mouse, were used. At birth, litters were randomly assigned to treatment with either saline or one of the three doses of the scAAV9-JeT-MCOLN1 vector. Injected mice were weaned and genotyped according to the standard procedure and subjected to open-field testing at the age of two months followed by euthanasia for tissue collection. qRT-PCR analysis revealed dose-dependent expression of the MCOLN1 transgene in the brain and peripheral tissues, with the highest overexpression in the cerebral cortex (FIG. 4A). ICV administration of the highest scAAV9-JeT-MCOLN1 dose prevented decline of motor deficits in the Mcoln1^{-/-} mice at the age of 2 months (FIGS. 4B, C). Post-mortem histological analysis of the brain tissue showed significant reduction in the density of lysosomal aggregates in the Mcoln1^{-/-} cerebral cortex as demonstrated on representative LAMP1 immunostaining images (FIG. 4D) and their quantification (FIG. 4E). LAMP1-positive lysosomal aggregates are a prominent pathological phenotype in the Mcoln1^{-/-} brain that can be measured either as a size of LAMP1-positive particles that are significantly enlarged in the Mcoln1^{-/-} mice, or as % of the LAMP1-positive area. Both measures were reduced by MCOLN1 gene transfer in the scAAV9-JeT-MCOLN1-treated Mcoln1^{-/-} mice (FIG. 4E). Importantly, histological analysis of brain myelination using immunostaining for the myelination marker Mbp showed increased Mbp-positive corpus callosum area in the scAAV9-JeT-MCOLN1-treated Mcoln1^{-/-} mice (FIG. 4F). Since developmental agenesis of the corpus callosum is a prominent feature of the human MLIV brain pathology, these data may have important implications for future MLIV gene therapy clinical trial using this vector design. In the cortical tissue, however, we did not observe significant increase in expression of Mbp with any of the doses of scAAV9-JeT-MCOLN1 used (FIG. 3A).

### Example 4. Neuron-Specific Expression of hMCOLN1 is Sufficient to Rescue Early Motor Dysfunction in MLIV Mice

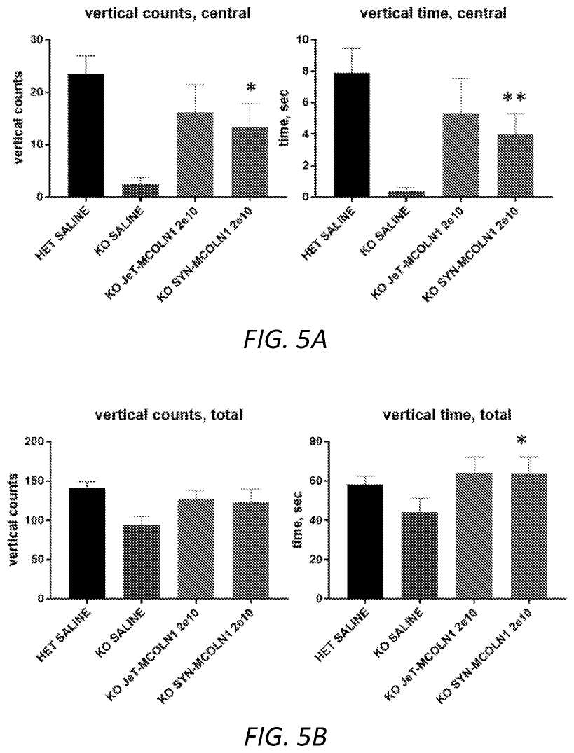

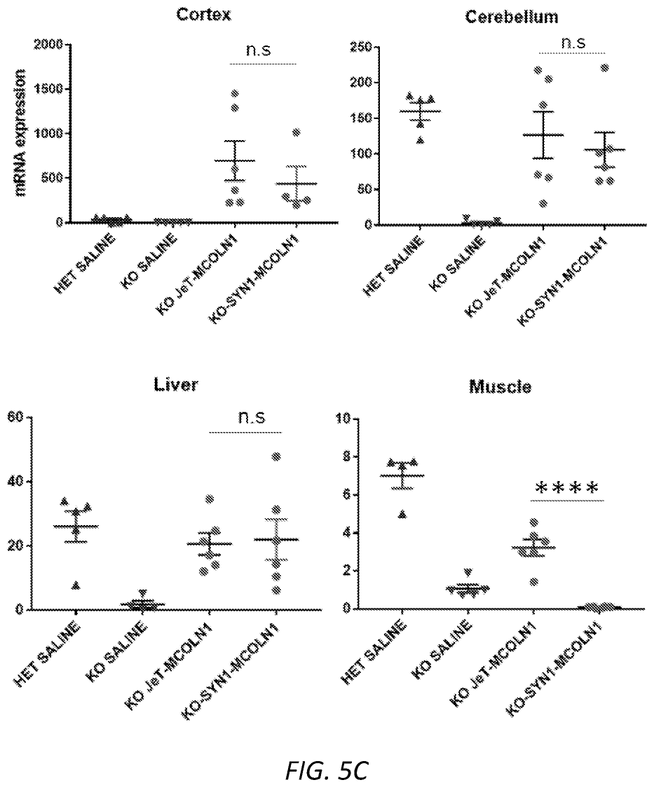

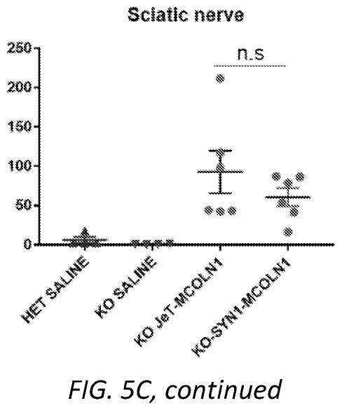

Neonatal intracerebroventricular delivery of scAAV9 vectors results in the widespread transduction of neurons and occasionally some glial cells in the mouse brain (15, 16) but also leads to transduction of peripheral organs, as shown by us (FIG. 4A) and others (16). To determine whether neuron-specific gene transfer of MCOLN1 is responsible for the therapeutic recovery of the neurologic function in the Mcoln1^{-/-} mouse model we next replaced the ubiquitously expressed JeT promoter with the neuron-specific human synapsin 1 (SYN1) promoter (16, 17) and administered the resulting scAAV9-SYN1-MCOLN1 vector to the postnatal day 1 mice via ICV route at the highest dose we used in the scAAV9-JeT-MCOLN1 experiment, 2×10¹⁰ vg/mouse, for direct comparison of the two vectors (Table 1). scAAV9-SYN1-MCOLN1-treated Mcoln1^{-/-} mice demonstrated motor function recovery similar to the scAAV9-JeT-MCOLN1-treated Mcoln1^{-/-} group (FIGS. 5A, B). Using MCOLN1 qRT-PCR for biodistribution analysis we found similar expression of the MCOLN1 transgene in the mouse brain tissue, cortex and cerebellum, as well as in periphery, in the liver and sciatic nerve (FIG. 5C). This is consistent with reports of SYN1 promoter driving expression in hepatocytes and PNS (16). As expected, no MCOLN1 expression was detected in the muscle tissue of the scAAV9-SYN1-MCOLN1-treated Mcoln1^{-/-} mice (FIG. 5C). Additionally, qRT-PCR analysis of the myelination marker Mbp showed no change in the Mbp transcripts level in the scAAV9-SYN1-MCOLN1-treated Mcoln1^{-/-} mice as compared to the saline-treated Mcoln1^{-/-} group. Similar to the scAAV9-JeT-MCOLN1 group, no difference in expression of microglial (C d 68) and astrocytic (Gfap) markers was observed in scAAV9-SYN1-MCOLN1-treated Mcoln1^{-/-} mice (Supplementary FIG. 3 B). No significant weight changes and overt health complications were observed in any of the cohorts treated with the scAAV9-MCOLN1 vectors during this study (Supplementary FIG. 3C). Overall these data indicate that: 1) CNS, but not muscle, targeting is critical to achieve therapeutic efficacy in MLIV; 2) within CNS, replacing MCOLN1 primarily in neurons is sufficient for motor function recovery, at least at the early symptomatic stage of the disease.

### Example 5. MCOLN1 Gene Transfer to Peripheral Organs Failed to Rescue Disease in Mcoln1^{-/-} Mice

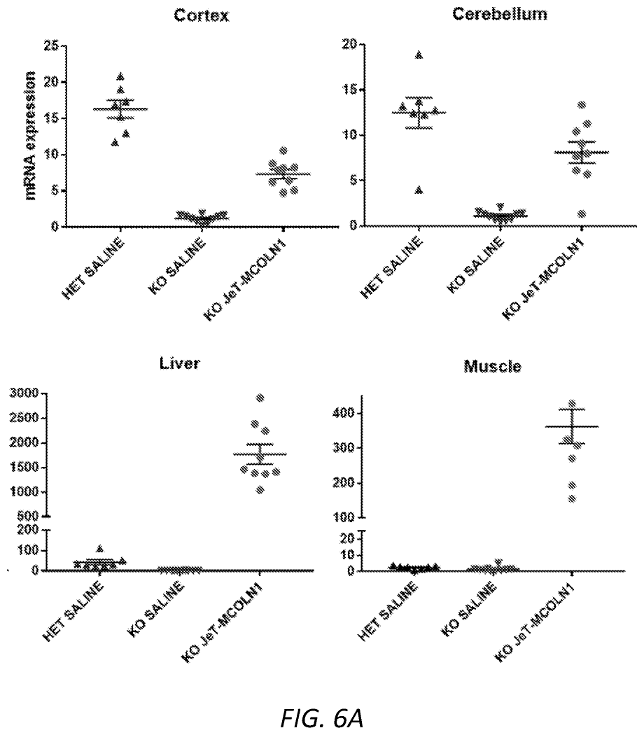

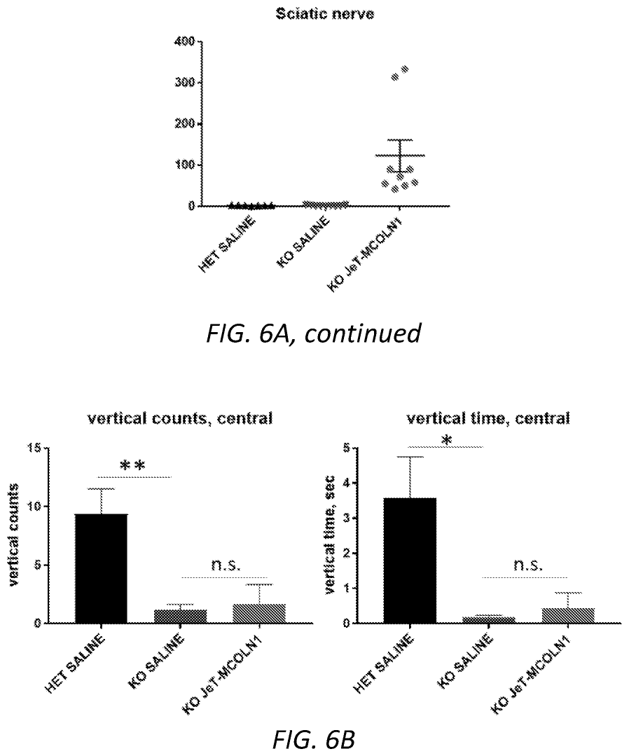

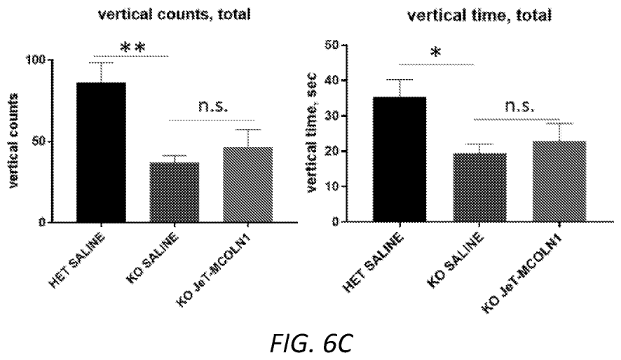

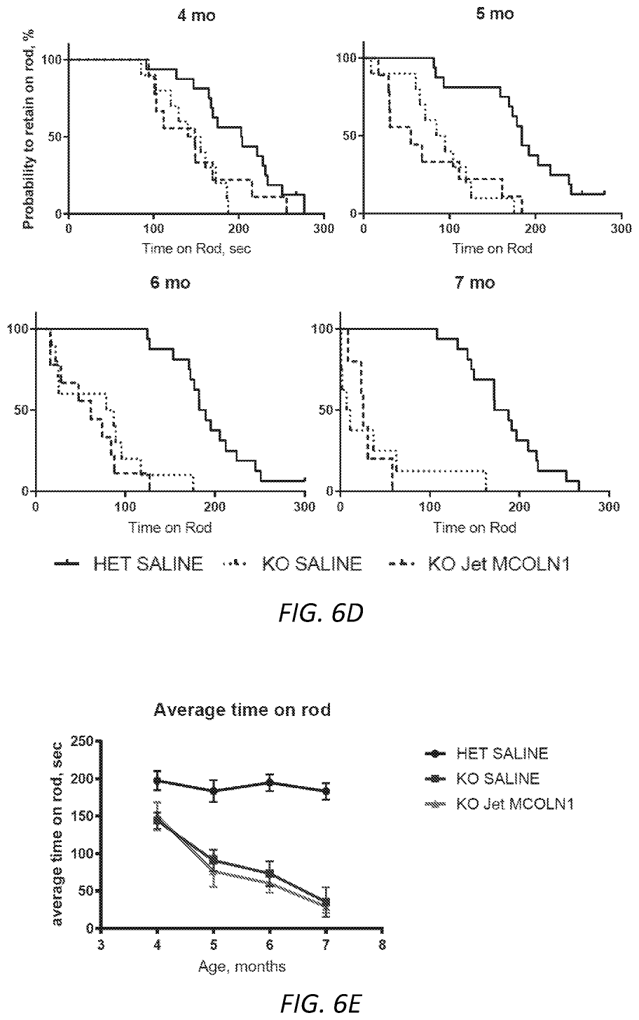

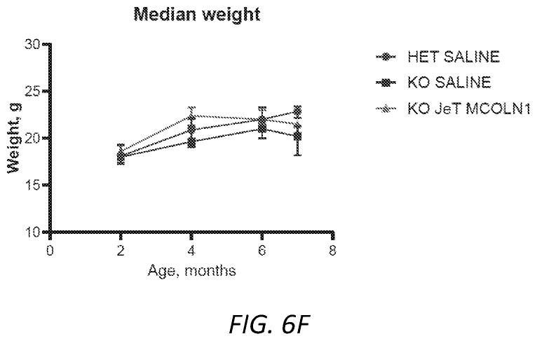

While MLIV most deleteriously affects CNS, endogenous MCOLN1 is expressed ubiquitously, and the pathological manifestations of its loss have been reported in peripheral organs, such as muscles, peripheral nerves and stomach (6, 7, 18-20). We next tested whether systemic administration of scAAV9-JeT-MCOLN1 in young adult symptomatic mice would have a therapeutic effect in Mcoln1^{-/-} mice. In this experiment Mcoln1^{-/-} and control Mcoln1^{+/-} mice were intravenously administered either saline or the scAAV9-JeT-MCOLN1 vector at the age of two months (Table 1). Motor function was assessed in the open field test once at the age of 4 months followed by rotarod testing monthly until the completion of the trial. The qRT-PCR analysis of MCOLN1 transgene expression showed very low expression in the brain and strong expression in peripheral organs such as skeletal muscle and sciatic nerve, with the highest expression values in the liver (FIG. 6A). Despite successful transgene expression in these tissues, we detected no motor function rescue in the scAAV9-JeT-MCOLN1-treated Mcoln1^{-/-} cohort as compared to saline-treated Mcoln1^{-/-} littermates in either open-field or rotarod tests from 4 to 7 months of age (FIGS. 6B, C, D, E) demonstrating that CNS gene transfer is critical to gain therapeutic efficacy. As in all previous trials with MCOLN1 transfer reported here, no significant weight changes or overt health impacts were noted in the cohort treated intravenously with scAAV9-JeT-MCOLN1 (FIG. 6F).

### REFERENCES

1. Grishchuk Y, Sri S, Rudinskiy N, Ma W, Stember K G, Cottle M W, et al. Behavioral deficits, early gliosis, dysmyelination and synaptic dysfunction in a mouse model of mucolipidosis IV. Acta neuropathologica communications. 2014; 2(1):133.
2. Levitt N, Briggs D, Gil A, Proudfoot N J. Definition of an efficient synthetic poly(A) site. Genes Dev. 1989; 3(7):1019-25.
3. Tornoe J, Kusk P, Johansen 1E, Jensen P R. Generation of a synthetic mammalian promoter library by modification of sequences spacing transcription factor binding sites. Gene. 2002; 297(1-2):21-32.
4. Challis R C, Ravindra Kumar S, Chan K Y, Challis C, Beadle K, Jang M J, et al. Systemic AAV vectors for widespread and targeted gene delivery in rodents. Nat Protoc. 2019; 14(2):379-414.
5. Zinn E, Pacouret S, Khaychuk V, Turunen H T, Carvalho L S, Andres-Mateos E, et al. In Silico Reconstruction of the Viral Evolutionary Lineage Yields a Potent Gene Therapy Vector. Cell reports. 2015; 12(6):1056-68.
6. Venugopal B, Browning M F, Curcio-Morelli C, Varro A, Michaud N, Nanthakumar N, et al. Neurologic, gastric, and opthalmologic pathologies in a murine model of mucolipidosis type IV. Am J Hum Genet. 2007; 81(5):1070-83.
7. Grishchuk Y, Pena K A, Coblentz J, King V E, Humphrey D M, Wang S L, et al. Impaired myelination and reduced brain ferric iron in the mouse model of mucolipidosis IV. Disease models & mechanisms. 2015; 8(12):1591-601.
8. Mepyans M, Andrzejczuk L, Sosa J, Smith S, Herron S, DeRosa S, et al. Early evidence of delayed oligodendrocyte maturation in the mouse model of mucolipidosis type IV. Disease models & mechanisms. 2020; 13(7).
9. Weinstock L, Furness A M, Herron S, Smith S S, Sankar S, DeRosa S G, et al. Fingolimod Phosphate Inhibits Astrocyte Inflammatory Activity in Mucolipidosis IV. Hum Mol Genet. 2018.
10. Hordeaux J, Wang Q, Katz N, Buza E L, Bell P, Wilson J M. The Neurotropic Properties of AAV-PHP.B Are Limited to C57BL/6J Mice. Molecular therapy: the journal of the American Society of Gene Therapy. 2018; 26(3):664-8.
11. Liguore W A, Domire J S, Button D, Wang Y, Dufour B D, Srinivasan S, et al. AAV-PHP.B Administration Results in a Differential Pattern of CNS Biodistribution in Non-human Primates Compared with Mice. Molecular therapy: the journal of the American Society of Gene Therapy. 2019; 27(11):2018-37.
12. Bailey R M, Armao D, Nagabhushan Kalburgi S, Gray S J. Development of Intrathecal AAV9 Gene Therapy for Giant Axonal Neuropathy. Mol Ther Methods Clin Dev. 2018; 9:160-71.
13. Meyer K, Ferraiuolo L, Schmelzer L, Braun L, McGovern V, Likhite S, et al. Improving single injection CSF delivery of AAV9-mediated gene therapy for SMA: a dose-response study in mice and nonhuman primates. Molecular therapy: the journal of the American Society of Gene Therapy. 2015; 23(3):477-87.
14. Mendell J R, Al-Zaidy S, Shell R, Arnold W D, Rodino-Klapac L R, Prior T W, et al. Single-Dose Gene-Replacement Therapy for Spinal Muscular Atrophy. N Engl J Med. 2017; 377(18): 1713-22.
15. Jackson K L, Dayton R D, Klein R L. AAV9 supports wide-scale transduction of the CNS and TDP-43 disease modeling in adult rats. Mol Ther Methods Clin Dev. 2015; 2:15036.
16. Jackson K L, Dayton R D, Deverman B E, Klein R L. Better Targeting, Better Efficiency for Wide-Scale Neuronal Transduction with the Synapsin Promoter and AAV-PHP.B. Front Mol Neurosci. 2016; 9:116.
17. DeGennaro L J, Kanazir S D, Wallace W C, Lewis R M, Greengard P. Neuron-specific phosphoproteins as models for neuronal gene expression. Cold Spring Harb Symp Quant Biol. 1983; 48 Pt 1:337-45.
18. Cheng X, Zhang X, Gao Q, Ali Samie M, Azar M, Tsang W L, et al. The intracellular Ca(2)(+) channel MCOLN1 is required for sarcolemma repair to prevent muscular dystrophy. Nat Med. 2014; 20(10):1187-92.
19. Chandra M, Zhou H, Li Q, Muallem S, Hofmann S L, Soyombo A A. A role for the Ca2+ channel TRPML1 in gastric acid secretion, based on analysis of knockout mice. Gastroenterology. 2011; 140(3):857-67.
20. Schiffmann R, Dwyer N K, Lubensky I A, Tsokos M, Sutliff V E, Latimer J S, et al. Constitutive achlorhydria in mucolipidosis type IV. Proc Natl Acad Sci USA. 1998; 95(3):1207-12.
21. Altarescu G., Sun M., Moore D. F., Smith J. A., Wiggs E. A., Solomon B. I., Patronas N. J., Frei K. P., Gupta S., Kaneski C. R. et al. (2002). The neurogenetics of mucolipidosis type IV. Neurology 59, 306-313.
22. Riedel K. G., Zwaan J., Kenyon K. R., Kolodny E. H., Hanninen L. and Albert D. M. (1985). Ocular abnormalities in mucolipidosis IV. Am. J. Ophthalmol. 99, 125-136.
23. Smith J. A., Chan C.-C., Goldin E. and Schiffmann R. (2002). Noninvasive diagnosis and ophthalmic features of mucolipidosis type IV. Ophthalmology 109, 588-594.
24. Wakabayashi K., Gustafson A. M., Sidransky E. and Goldin E. (2011). Mucolipidosis type IV: an update. Mol. Genet. Metab. 104, 206-213.
25. Chen C C, Keller M, Hess M, Schiffmann R, Urban N, Wolfgardt A, et al. A small molecule restores function to TRPML1 mutant isoforms responsible for mucolipidosis type IV. Nature communications. 2014; 5:4681.
26. Medina D L, Di Paola S, Peluso I, Armani A, De Stefani D, Venditti R, et al. Lysosomal calcium signalling regulates autophagy through calcineurin and TFEB. Nat Cell Biol. 2015; 17(3):288-99.
27. Settembre C, Medina D L. TFEB and the CLEAR network. Methods Cell Biol. 2015; 126:45-62.
28. Zhang X, Cheng X, Yu L, Yang J, Calvo R, Patnaik S, et al. MCOLN1 is a ROS sensor in lysosomes that regulates autophagy. Nature communications. 2016; 7:12109.
29. Wang W, Gao Q, Yang M, Zhang X, Yu L, Lawas M, et al. Up-regulation of lysosomal TRPML1 channels is essential for lysosomal adaptation to nutrient starvation. Proc Natl Acad Sci USA. 2015; 112(11):E1373-81.
30. Zhang X, Chen W, Gao Q, Yang J, Yan X, Zhao H, et al. Rapamycin directly activates lysosomal mucolipin TRP channels independent of mTOR. PLoS Biol. 2019; 17(5):e3000252.
31. Scotto Rosato A, Montefusco S, Soldati C, Di Paola S, Capuozzo A, Monfregola J, et al. TRPML1 links lysosomal calcium to autophagosome biogenesis through the activation of the CaMKKbeta/VPS34 pathway. Nat Commun. 2019; 10(1):5630.
32. Dong X P, Cheng X, Mills E, Delling M, Wang F, Kurz T, et al. The type IV mucolipidosis-associated protein TRPML1 is an endolysosomal iron release channel. Nature. 2008; 455(7215):992-6.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An adeno-associated (scAAV) viral vector comprising a sequence encoding Mucolipin-1 (MCOLN1) protein, operably linked to a promoter that drives expression of the MCOLN1 protein in a cell.

2. The AAV of claim 1, which is a self-complementary scAAV or single-stranded AAV-PHP.b.

3. The AAV of claim 1, further comprising one or more cis-regulatory elements that increase expression of the MCOLN1.

4. The AAV of claim 3, wherein the cis-regulatory elements comprise one or more enhancers; posttranscriptional regulatory elements; cell penetrating peptides; polyadenylation sequences; and/or an intron.

5. The AAV of claim 4, wherein the posttranscriptional regulatory element is HBV Posttranscriptional Regulatory Element (HPRE) or woodchuck hepatitis virus post-transcriptional regulatory element (WPRE), or a variant thereof.

6. The AAV of claim 4, wherein the intron is SV40 intron, F.IX truncated intron 1; β-globin SD/immunoglobin heavy chain SA; Adenovirus SD/immunoglobulin SA; SV40 late SD/SA (19S/16S); Hybrid adenovirus SD/IgG SA; or minute virus of mice (MVM) intron.

7. The AAV of claim 1, wherein the MCOLN1 protein is at least 80% identical to SEQ ID NO:1.

8. The AAV of claim 1, wherein the promotor is a ubiquitous promoter, preferably Jet promoter, or a neuron-specific promoter, preferably a synapsin I (Syn1) promoter.

9. The AAV of claim 1, wherein the AAV is AAV9 serotype and comprises a JeT promoter or a Syn1 promoter and polyadenylation sequence.

10. A method of treating mucolipidosis IV in a subject, the method comprising administering to the subject a therapeutically effective amount of the AAV of claim 1.

11. The method of claim 10, wherein the AAV is administered to the subject by direct administration into the CNS or eye of the subject.

12. The method of claim 11, wherein administration into the CNS is by intrathecal or intracerebroventicular injection.

13. The method of claim 10, wherein the subject has developed one or more symptoms of mucolipidosis IV selected from spasticity, hypotonia, an inability to walk independently, ptosis, myopathic facies, drooling, difficulties in chewing and swallowing, impaired fine-motor function, and progressive blindness.

14. The method of claim 13, wherein the one or more symptoms of mucolipidosis IV are improved.

15. The method of claim 10, wherein the AAV is administered once, twice, three times, or more.

16.-21. (canceled)

22. The method of claim 15, wherein the AAV is administered once weekly, once monthly, biweekly, bimonthly, every three months, every four months, every five months, every six months, or annually.
